(19) United States
(12) Patent Application Publication
(10) Pub. No.: US 2011/0287135 A1
(43) Pub. Date: Nov. 24, 2011

(54) NOVEL ARABINOHYDROLASES

(76) Inventors: Stefan Kuehnel, Wageningen (NL); Henk A. Schols, Wageningen (NL); Yvonne Westphal, Wageningen (NL); Sandra Hinz, Wageningen (NL); Jacob Visser, Wageningen (NL)

(21) Appl. No.: 13/024,184

(22) Filed: Feb. 9, 2011

Related U.S. Application Data
(63) Continuation-in-part of application No. 12/205,694, filed on Sep. 5, 2008.
Application No. 12/205,694 is a continuation-in-part of application No. 11/833,133, filed on Aug. 2, 2007, now Pat. No. 7,923,236.
(60) Provisional application No. 61/302,882, filed on Feb. 9, 2010.

(51) Int. Cl. C12P 19/00; A23L 2/02; C12N 9/24; C12G 1/00; C12P 19/02; C12N 1/15

(52) U.S. Cl. 426/15; 435/105; 435/254.11; 435/72; 435/200; 426/51

(57) ABSTRACT

The invention relates to enzymes, compositions, and methods for efficient hydrolysis of arabinans present in plant biomass. More specifically, the invention relates to arabinases and compositions comprising arabinases to improve cell wall degradation to efficiently use plant biomass for bio-energy production. The invention also relates to a method for preparing a prebiotic. The prebiotic may contain branched arabinan oligomers comprise α-(1,5)-linked arabinan backbone, and single substituted α-(1,3)-linked arabinose monomers attached to the backbone, or double substituted α-(1,2,3,5)-linked arabinose monomers attached to the backbone, or both. The invention also relates to a method for preparing fruit juice or wine. The invention also relates to a method for saccharification of a plant biomass. The invention also relates to a recombinant micro-organism genetically modified to express the enzymes of the present invention and optionally additional enzymes to achieve the disclosed methods.

This application claims priority under 35 U.S.C. §119 to U.S. Provisional Patent Application Ser. No. 61/302,882, filed Feb. 9, 2010, the contents of which are hereby incorporated by reference in their entirety. This application is also a continuation-in-part of U.S. patent application Ser. No. 11/833,133, filed Aug. 2, 2007, and a continuation-in-part of and U.S. patent application Ser. No. 12/205,694, filed Sep. 5, 2008. Each of these applications is hereby incorporated by reference in its entirety.

### FIELD OF THE INVENTION

The present invention relates to enzymes, compositions, and methods for efficient hydrolysis of arabinans present in plant biomass. The invention provides amongst other things, arabinases and compositions comprising arabinases to improve cell wall degradation to efficiently use plant biomass for bio-energy production. The invention also provides methods for preparing a prebiotic. The prebiotic may contain branched arabinan oligomers comprise α-(1,5)-linked arabinan backbone, and single substituted α-(1,3)-linked arabinose monomers attached to the backbone, or double substituted α-(1,2,3,5)-linked arabinose monomers attached to the backbone, or both. The invention also provides methods for preparing fruit juice or wine. The invention also provides a method for saccharification of a plant biomass. The invention also provides among other things, a recombinant micro-organism genetically modified to express the enzymes of the present invention and optionally additional enzymes to achieve the disclosed methods.

### BACKGROUND OF THE INVENTION

Large amounts of carbohydrates in plant biomass provide a plentiful source of potential energy in the form of sugars (both five carbon and six carbon sugars) that can be utilized for numerous industrial and agricultural processes. Sugars generated from degradation of plant biomass potentially represent plentiful, economically competitive feedstocks for fermentation into chemicals, plastics, and fuels, including ethanol as a substitute for petroleum. However, the enormous energy potential of these carbohydrates is currently under-utilized because the sugars are locked in complex polymers, and hence are not readily accessible for fermentation.

Pectins are one of the main complex polymers within the primary plant cell wall. Four main pectic components have been identified: homogalacturonan (HG), rhamnogalacturonan I (RG I), rhamnogalacturonan II (RG II) and xylogalacturonan (XGA) which have been described extensively (Ralet and Thibault, 2002; Ridley, 2001; Voragen, 1995). The rhamnogalacturonan I may be branched and the side chains may comprise neutral sugar chains, like arabinan or galactan. Pectic arabinan is a branched molecule with a linear α-(1,5)-linked arabinose backbone which can be single or double substituted with α-(1,2)-linked and/or α-(1,3)-linked arabinose side chains which again may be further branched (Beldman, 1997; Weinstein and Albersheim, 1979). In addition, the arabinan of e.g. sugar beet cell walls has been shown to be feruloylated at the O-2 and/or O-5 position (Levigne, 2004).

A number of different enzymes are known to degrade arabinans. Endoarabinanases are endo-acting enzymes that hydrolyze the linear regions of the arabinan backbone and release a mixture of arabinose and arabinose oligomers (Beldman et al., 1997). All other arabinose releasing enzymes release arabinose from the non-reducing end (Chávez Montes et al., 2008). Exoarabinanases release arabinose (Ichinose et al., 2008), arabinobiose (Carapito et al., 2009; Sakamoto and Thibault, 2001) or arabinotriose (Kaji, 1984) from linear α-1,5-linked arabinan. Arabinofuranosidases (Abf) subgroup into A and B. Abf A is active towards arabinose oligomers and p-NP-arabinofuranoside, but does not act on polymers. Abf A can hydrolyze all kinds of linkages present in arabinan and arabinoxylan oligomers (Matsuo et al., 2000). Abf B is active towards p-NP-arabinofuranoside and beet arabinan polymers. Abf B acts mainly on α-1,3-linked arabinose and much less on α-1,5-linkages (Rombouts et al., 1988). Some Abf B also show activity towards arabinoxylan oligomers (de Vries and Visser, 2001). Although arabinoxylan arabinofuranohydrolases (AXH) release arabinose specifically from arabinoxylan and some AXH also degrade arabinan (de Vries and Visser, 2002; Kormelink et al., 1991).

Complete cell wall degradation is required to efficiently use plant biomass for bio-energy production. However, currently available enzyme preparations do not lead to an efficient hydrolysis of arabinans. Thus, there exists a need in the art for enzyme preparations that can solubilize arabinan efficiently and provide greater yields of arabinose.

The invention described herein addresses this need by providing compositions and methods for efficient hydrolysis of arabinans present in a plant biomass, using sugar beet pulp as an example of plant biomass. Sugar beet pulp is a major byproduct of sugar production from beet that remains after extraction of the sugar beet roots. The dried sugar beet pulp has a total carbohydrate content of 75% of which glucose and arabinose are the predominant monosaccharides, present as part of the cell wall polysaccharides cellulose and pectin, respectively (McCready, 1966). Pectic sugar beet arabinans represent 20-25% of the sugar beet pulp dry matter. Commercial enzyme preparations can solubilize arabinan from sugar beet pulp with monomer yields of only up to 67% (Micard et al., 1996). Due to the complex, interwoven structure of the cell wall, a more efficient release of arabinan may also require cellulase activities, which are lacking in the commercial preparations (Micard et al., 1996). The ascomycete Chrysosporium lucknowense C1 is an industrial strain optimized in cellulase and hemicellulase production which seems to be a good platform for the degradation of pectin rich biomass.

### SUMMARY OF THE INVENTION

The present invention provides a method for hydrolyzing arabinans present in a plant biomass, comprising contacting the plant biomass with a multi-enzyme composition, wherein the multi-enzyme composition is selected from the group consisting of:
a. Abn1 (SEQ ID NO:2), Abn2 (SEQ ID NO:4), Abn4 (SEQ ID NO:6), and Abf3 (SEQ ID NO:8);
b. Abn1 (SEQ ID NO:2), Abn2 (SEQ ID NO:4), and Abn4 (SEQ ID NO:6); and
c. Abn1 (SEQ ID NO:2), Abn4 (SEQ ID NO:6) and Abf3 (SEQ ID NO:8);

In some embodiments, the multi-enzyme composition is able to degrade at least about 70%, at least about 80%, or at least about 90% of the arabinan present in the plant biomass to arabinose.

In some embodiments, the enzymes are isolated from a filamentous fungus.

In some embodiments, the specific activity of Abn1 towards linear arabinan is from about 20 U/mg to about 30 U/mg, the specific activity of Abn2 towards linear arabinan is from about 6 U/mg to about 8 U/mg, the specific activity of Abn4 towards branched arabinan is from about 8 U/mg to about 11 U/mg, and the specific activity of Abf3 towards p-Nitrophenyl-α-arabinofuranose is from about 20 U/mg to about 30 U/mg.

The invention also provides a multi-enzyme composition comprising the enzymes Abn1 (SEQ ID NO:2), Abn2 (SEQ ID NO:4), Abn4 (SEQ ID NO:6), and Abf3 (SEQ ID NO:8) wherein the multi-enzyme composition is able to degrade at least about 70%, at least about 80%, or at least about 90% of the arabinan present in sugar beet to arabinose.

The invention also provides a multi-enzyme composition comprising the enzymes Abn1 (SEQ ID NO:2), Abn2 (SEQ ID NO:4), and Abn4 (SEQ ID NO:6) wherein the multi-enzyme composition is able to degrade at least about 70%, at least about 80%, or at least about 90% of the arabinan present in sugar beet to arabinose.

The invention also provides a multi-enzyme composition comprising the enzymes Abn1 (SEQ ID NO:2), Abn4 (SEQ ID NO:6), and Abf3 (SEQ ID NO:8) wherein the multi-enzyme composition is able to degrade at least about 70%, at least about 80%, or at least about 90% of the arabinan present in sugar beet to arabinose.

The invention also provides a method for preparing a prebiotic comprising contacting a plant biomass comprising arabinans with a multi-enzyme composition, wherein the multi-enzyme composition comprises Abn1 (SEQ ID NO:2), Abn2 (SEQ ID NO:4), and Abn4 (SEQ ID NO:6) and wherein the multi-enzyme composition is capable of degrading the arabinans in the plant biomass into linear and branched arabinanose oligomers.

The invention also provides a multi-enzyme composition useful in the preparation of a prebiotic comprising the enzymes Abn1 (SEQ ID NO:2), Abn2 (SEQ ID NO:4), and Abn4 (SEQ ID NO:6), wherein the multi-enzyme composition is able to hydrolyze arabinan present in the plant biomass into branched arabinan oligomers.

In some embodiments, the prebiotic comprises branched arabinan oligomers, wherein the branched arabinan oligomers comprise α-(1,5)-linked arabinan backbone, and a) single substituted α-(1,3)-linked arabinose monomers attached to the backbone, or b) double substituted α-(1,2,3,5)-linked arabinose monomers attached to the backbone, c) or both.

In some embodiments, the branched arabinan oligomers comprise α-(1,5)-linked arabinan backbone, and a) single substituted α-(1,3)-linked arabinose monomers attached to the backbone, or b) double substituted α-(1,2,3,5)-linked arabinose monomers attached to the backbone, c) or both.

The invention also provides a method for preparing a fruit juice or wine comprising contacting a plant biomass with a multi-enzyme composition, wherein the multi-enzyme composition comprises Abn1 (SEQ ID NO:2), Abn2 (SEQ ID NO:4), Abn4 (SEQ ID NO:6) and Abf3 (SEQ ID NO:8) and wherein the multi-enzyme composition is capable of degrading the arabinans in the plant biomass into linear and branched arabinanose oligomers.

The invention also provides a method for saccharification of a plant biomass comprising contacting the plant biomass with a multi-enzyme composition, wherein the multi-enzyme composition comprises Abn1 (SEQ ID NO:2), Abn2 (SEQ ID NO:4), Abn4 (SEQ ID NO:6,) and Abf3 (SEQ ID NO:8) and wherein the multi-enzyme composition is capable of degrading the arabinans in the plant biomass into linear and branched arabinanose oligomers.

In some embodiments, the multi-enzyme composition further comprises one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase and wherein the plant biomass comprises pectins, hemi-celluloses and/or celluloses.

The invention also provides a recombinant micro-organism, wherein the microorganism is genetically modified to express Abn1 (SEQ ID NO:2), Abn2 (SEQ ID NO:4), Abn4 (SEQ ID NO:6), Abf3 (SEQ ID NO:8), or a combination thereof.

In some embodiments, the micro-organism expresses one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase.

In some embodiments, the micro-organism is a filamentous fungus.

These and other embodiments are disclosed or are apparent from and encompassed by the following Detailed Description.

### BRIEF DESCRIPTION OF THE DRAWINGS

FIG. 1: Biochemical characterization of Abn1, Abn2 and Abn4. A) pH optima, B) temperature optima, C) pH stabilities, D) temperature stabilities. Activities are determined on linear arabinan (Abn1 and Abn2) and branched arabinan (Abn4), respectively. (n=3)

FIG. 2: The degradation of linear arabinose oligomers by C1 arabinohydrolases determined by HPAEC. X-axis: Arabinose oligomers DP1-6 used as substrate. A) Abn1, B) Abn2, C) Abn4

FIG. 3: HPSEC elution patterns of linear (A) and branched (B) arabinans digested with different combinations of C1 arabinohydrolases. Elution times of pullulan standards are indicated.

FIG. 4: Arabinose oligomers release from linear (A) and branched (B) arabinan with different combinations of C1 arabinohydrolases as determined by HPAEC. X-axis: released monomers and oligomers from DP2-6 and total release (Sum).

FIG. 5: Release of non linear arabinose oligomers from branched arabinan by C1 arabinohydrolases as determined by HPAEC. A) Default HPAEC gradient with total sugar concentrations between 50 and 100 μg/ml. Line a—Abn2; line b—Abn1 and Abn4; line c—Abn1, Abn2 and Abn4. B) Less steep HPAEC gradient with total sugar concentrations of 500 to 1000 μg/ml. Line a—branched arabinan blank; line b—Abn1, Abn2 and Abn4; line c—Abn1, Abn2, Abn4 and Afb3. Ara1 to Ara6: retention times of linear arabinose oligomers with DP1-6. Asterisks indicate peaks of unknown structure.

FIG. 6: HPAEC elution pattern of AOS after degradation of sugar beet arabinan with different amounts of Abn4 followed by end-point-incubation with Abn1 and Abn2: D-30 (A), D-100 (B); indication of linear α-(1,5)-linked AOS (DP1-7).

FIG. 7: Biogel P2 elution pattern of the D-30 digest with indication of the pooled fractions and their degree of polymerization (DP) as analyzed by MALDI-TOF MS (A); HPAEC elution pattern of pooled fractions (B; zoom) with indication of linear α-(1,5)-linked AOS (DP3-6); inserted table represents the DP of the fractions as analyzed with MALDI-TOF MS.

FIG. 8: Biogel P2 elution pattern of the D-100 digest with indication of the pooled fractions and their degree of polymerization (DP) as analyzed by MALDI-TOF MS (A); HPAEC elution pattern of pooled fractions (B; zoom) with indication of linear α-(1,5)-linked AOS (DP3-8); inserted table represents the DP of the fractions as analyzed with MALDI-TOF MS.

FIG. 9: [¹H,¹³C]-HMBC spectrum of pool III₃₀ (zoom at 5.40-5.00 ppm (¹H) and 66-90 ppm (¹³C), respectively); T and A as indicated in Table 3.

FIG. 10: [¹H,¹³C]-HMBC spectrum of pool V₁₀₀ (zoom at 5.40-5.00 (¹H) and 66-90 (¹³C), respectively); Tₙ, A, B and C as indicated in Table 3.

### DETAILED DESCRIPTION OF THE INVENTION

Definitions

As used herein:

“α-L-arabinofuranosidase”, “α-N-arabinofuranosidase”, “α-arabinofuranosidase”, “arabinosidase” or “arabinofuranosidase” refers to a protein that hydrolyzes arabinofuranosyl-containing hemicelluloses. Some of these enzymes remove arabinofuranoside residues from O-2 or O-3 single substituted xylose and/or arabinose residues, as well as from O-2 and/or O-3 double substituted xylose and/or arabinose residues.

“Endo-arabinase” refers to a protein that catalyzes the hydrolysis of 1,5-α-arabinofuranosidic linkages in 1,5-arabinans, producing arabinooligosaccharides.

“Exo-arabinase” refers to a protein that catalyzes the hydrolysis of 1,5-α-linkages in 1,5-arabinans or 1,5-α-L-arabino-oligosaccharides, releasing mainly arabinose and/or arabinobiose, although a small amount of arabinotriose can also be liberated.

“Xylanase” specifically refers to an enzyme that hydrolyzes the .beta.-1,4 bond in the xylan backbone, producing short xylooligosaccharides.

“carbohydrase” refers to any protein that catalyzes the hydrolysis of carbohydrates. Endoglucanases, cellobiohydrolases, β-glucosidases, α-glucosidases, xylanases, β-xylosidases, galactanases, α-galactosidases, β-galactosidases, α-amylases, glucoamylases, endo-arabinases, arabinofuranosidases, mannanases, β-mannosidases, pectinases, acetyl xylan esterases, acetyl mannan esterases, ferulic acid esterases, coumaric acid esterases, pectin methyl esterases, and chitosanases are examples of glycosidases.

“Hemicellulase” refers to a protein that catalyzes the hydrolysis of hemicellulose, such as that found in lignocellulosic materials. Hemicelluloses are complex polymers, and their composition often varies widely from organism to organism, and from one tissue type to another. Hemicelluloses include a variety of compounds, such as xylans, arabinoxylans, xyloglucans, mannans, glucomannans, and galactomannans. Hemicellulose can also contain glucan, which is a general term for beta-linked glucose residues. In general, a main component of hemicellulose is beta-1,4-linked xylose, a five carbon sugar. However, this xylose is often substituted with alpha-1,3 linked or alpha-1,2 linked arabinose or glucuronic acid, which can be substituted by beta-1,2 galactose, mannose, and/or xylose, or by ferulic acid residues. The xylose residues in the backbone can also be esterified to acetic acid. The composition, nature of substitution, and degree of branching of hemicellulose is very different in dicotyledonous plants (dicots, i.e., plant whose seeds have two cotyledons or seed leaves such as lima beans, peanuts, almonds, peas, kidney beans) as compared to monocotyledonous plants (monocots; i.e., plants having a single cotyledon or seed leaf such as corn, wheat, rice, grasses, barley). In dicots, hemicellulose is comprised mainly of xyloglucans that are 1,4-beta-linked glucose chains with 1,6-alpha-linked xylosyl side chains. In monocots, including most grain crops, the principal components of hemicellulose are heteroxylans. These are primarily comprised of 1,4-beta-linked xylose backbone polymers with 1,2- or 1,3-alpha/beta linkages to arabinose, glucuronic acid, galactose and mannose as well as xylose modified by ester-linked acetic acids. Also present are branched beta glucans comprised of 1,3- and 1,4-beta-linked glucosyl chains. In monocots, cellulose, heteroxylans and beta glucans are present in roughly equal amounts, each comprising about 15-25% of the dry matter of cell walls. “Hemicellulase” refers to a protein that catalyzes the hydrolysis of hemicellulose, such as that found in lignocellulosic materials. Hemicellulose is a complex polymers, and their composition often varies widely from organism to organism, and from one tissue type to another. Hemicellulolytic enzymes, i.e. hemicellulases, include both endo-acting and exo-acting enzymes, such as xylanases, β-xylosidases, galactanases, α-galactosidases, β-galactosidases, endo-arabinases, arabinofuranosidases, mannanases, β-mannosidases. Hemicellulases also include the accessory enzymes, such as alpha-glucuronidases, acetylesterases, glucuronyl esterases, ferulic acid esterases, and coumaric acid esterases. Among these, xylanases and acetyl xylan esterases cleave the xylan and acetyl side chains of xylan and the remaining xylo-oligomers are unsubstituted and can thus be hydrolysed with β-xylosidase only. In addition, several less known side activities have been found in enzyme preparations which hydrolyze hemicellulose. Accordingly, xylanases, acetylesterases and β-xylosidases are examples of hemicellulases. Similarly the other accessory enzymes mentioned remove glucuronic acid, ferulic acid and coumaric acid which also form obstacles for complete degradation of the hemicellulose structure.

“β-Mannanase” or “endo-1,4-β-mannosidase” refers to a protein that hydrolyzes mannan-based hemicelluloses (mannan, glucomannan, galactomannan) and produces short β-1,4-mannooligosaccharides.

“Mannan endo-1,6-α-mannosidase” refers to a protein that hydrolyzes 1,6-α-mannosidic linkages in unbranched 1,6-mannans.

“β-Mannosidase” (β-1,4-mannoside mannohydrolase; EC 3.2.1.25) refers to a protein that catalyzes the removal of β-D-mannose residues from the nonreducing ends of oligosaccharides.

“Galactanase”, “endo-β-1,6-galactanse” or “arabinogalactan endo-1,4-β-galactosidase” refers to a protein that catalyzes the hydrolysis of endo-1,4-β-D-galactosidic or endo-1,6-β-D-galactosidic linkages in arabinogalactans.

“β-xylosidase” refers to a protein that hydrolyzes short 1,4-β-D-xylooligomers into xylose.

“α-Glucuronidase” refers to a protein that hydrolyzes the 1,2-α-glucuronic acid linkages in hemicelluloses.

“Acetyl xylan esterase” refers to a protein that catalyzes the removal of the acetyl groups from xylose residues. “Acetyl mannan esterase” refers to a protein that catalyzes the removal of the acetyl groups from mannose residues. “feruloyl esterase” or “ferulic acid esterase” refers to a protein that hydrolyzes the ester bond between the arabinose substituent group and ferulic acid. “Coumaric acid esterase” refers to a protein that hydrolyzes the ester bond between the arabinose substituent group and coumaric acid.

“Glucuronyl esterase” refers to a protein that hydrolyzes the ester bond between glucuronic acid and lignin. Acetyl xylan esterases, glucuronyl esterases, ferulic acid esterases and coumaric acid esterases are examples of carbohydrate esterases.

“Pectin” refers to polysaccharides which are composed of homogalacturonan and rhamnogalacturonan. Homogalacturonan is composed of alpha 1,4-linked galacturonic acid residues which may be methyl esterified at the C6 carboxylate function and/or acetylated at the C2 or C3 position.

Rhamnogalacturonan is composed of alternating α-1,2-rhamnose and α-1,4-linked galacturonic acid, with side chains linked 1,4 to rhamnose. The side chains include Type I galactan, which is β-1,4-linked galactose with α-1,3-linked arabinose substituents; Type II galactan, which is β-1,3-1,6-linked galactoses (very branched) with arabinose substituents; and arabinan, which is α-1,5-linked arabinose with α-1,3-linked or α-1,2-linked arabinose branches. The galacturonic acid substituents may be acetylated and/or methylated.

Pectinolytic enzymes include both endo-acting and exo-acting enzymes, such as polygalacturonases, pectin and pectate lyases, arabinofuranosidases, rhamnosidases and several esterases like pectin methyl esterases. These and some other enzymes found like ferulic acid esterases are suitable to be used in multi-enzyme compositions to degrade pectin materials.

“Pectin methyl esterase” refers to a protein that catalyzes the removal of the methyl groups ester linked to the carboxylic acid residues in galacturonic acid

“Rhamnogalacturonan acetylesterase” refers to a protein that catalyzes the removal of the acetyl groups ester-linked to the highly branched rhamnogalacturonan (hairy) regions of pectin.

“Pectin acetyl esterase” refers to a protein that catalyzes the removal of the acetyl groups ester-linked to the homogalacturonan (smooth) regions of pectin.

Esterases active on pectin are another examples of carbohydrate esterases.

“Polygalacturonase” refers to a protein that catalyzes the hydrolysis of alpha 1,4-linked galacturonic acid residues from homogalacturonan thus converting polygalacturonides to galacturonic acid or galacturonic acid oligosaccharides.

“Rhamnogalacturon hydrolase” refers to a protein that catalyzes the degradation of the rhamnogalacturonan backbone of pectin to galacturonic acid or rhamnogalacturonan oligosaccharides.

“Pectate lyase” and “pectin lyases” refer to proteins that catalyze the cleavage of 1,4-α-D-galacturonan by beta-elimination acting on polymeric and/or oligosaccharide substrates (pectates and pectins, respectively).

“Pectate lyase” refers to a protein that catalyzes the cleavage of 1,4-α-D-galacturonan by beta-elimination acting on polymeric and/or oligosaccharide substrates.

“Pectin lyase” refers to a protein that catalyzes the cleavage of 1,4-α-D-galacturonan by beta-elimination acting on polymeric and/or oligosaccharide substrates. The action of the enzyme is not hindered by acetyl esters.

“Rhamnogalacturonan lyase” refers to a protein that catalyzes the degradation of the rhamnogalacturonan backbone of pectin via a β-elimination mechanism (see, e.g., Pages et al., J. Bacteria. 185:4727-4733 (2003)).

Glycosidases (glycoside hydrolases; GH), a large family of enzymes that includes cellulases and hemicellulases, catalyze the hydrolysis of glycosidic linkages, predominantly in carbohydrates. Glycosidases such as the proteins of the present invention may be assigned to families on the basis of sequence similarities, and there are now over 100 different such families defined (see the CAZy (Carbohydrate Active EnZymes database) website, maintained by the Architecture of Fonction de Macromolecules Biologiques of the Centre National de la Recherche Scientifique, which describes the families of structurally-related catalytic and carbohydrate-binding modules (or functional domains) of enzymes that degrade, modify, or create glycosidic bonds; Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12). Because there is a direct relationship between the amino acid sequence of a protein and its folding similarities, such a classification reflects the structural features of these enzymes and their substrate specificity. Such a classification system can help to reveal the evolutionary relationships between these enzymes and provide a convenient tool to determine information such as an enzyme's activity and function. Thus, enzymes assigned to a particular family based on sequence homology with other members of the family are expected to have similar enzymatic activities and related substrate specificities. CAZy family classifications also exist for glycosyltransferases (GT), polysaccharide lyases (PL), and carbohydrate esterases (CE). Likewise, sequence homology may be used to identify particular domains within proteins, such as carbohydrate binding modules (CBMs; also known as carbohydrate binding domains (CBDs)). An enzyme assigned to a particular CAZy family may exhibit one or more of the enzymatic activities or substrate specificities associated with the CAZy family. In other embodiments, the enzymes of the present invention may exhibit one or more of the enzyme activities discussed above.

“Overdose” refers to a concentration of enzyme and substrate where there is more enzyme than available substrate. At that concentration there is an overdose.

Enzymes and Compositions of the Invention

As described herein, a novel multi-enzyme composition comprising at least one of the enzymes endoarabinanase Abn1 (nucleic acid: SEQ ID NO:1; amino acid: SEQ ID NO:2), exoarabinanase Abn2 (nucleic acid: SEQ ID NO:3; amino acid: SEQ ID NO:4), arabinofuranosidase (Abn4) (nucleic acid: SEQ ID NO:5; amino acid: SEQ ID NO:6) and arabinoxylan arabinofuranohydrolase Abf3 (nucleic acid: SEQ ID NO:7; amino acid: SEQ ID NO:8), is capable of effecting complete or nearly complete degradation of arabinan present in a plant biomass. These enzymes were first described in U.S. patent application Ser. Nos. 11/833,133 and 12/205,694, both of which are incorporated by reference herein.) For example, the multi-enzyme composition was able to hydrolyze about 80% of the arabinan present in the sugar beet pulp to fermentable monosugars.

The enzyme Abn1 is encoded by the nucleic acid sequence represented herein as SEQ ID NO:1 and the cDNA sequence represented herein as SEQ ID NO:9. The Abn1 nucleic acid sequence encodes a 321 amino acid sequence, represented herein as SEQ ID NO:2. The signal peptide for Abn1 is located from positions 1 to about position 20 of SEQ ID NO:2, with the mature protein spanning from about position 21 to position 321 of SEQ ID NO:2. Within Abn1 is a catalytic domain (CD). The amino acid sequence containing the CD of Abn1 spans from a starting point of about position 27 of SEQ ID NO:2 to an ending point of about position 321 of SEQ ID NO:2.

The enzyme Abn2 is encoded by the nucleic acid sequence represented herein as SEQ ID NO:3 and the cDNA sequence represented herein as SEQ ID NO:10. The Abn2 nucleic acid sequence encodes a 378 amino acid sequence, represented herein as SEQ ID NO:4. The signal peptide for Abn2 is located from positions 1 to about position 17 of SEQ ID NO:4, with the mature protein spanning from about position 18 to position 378 of SEQ ID NO:4. Within Abn2 is a catalytic domain (CD). The amino acid sequence containing the CD of Abn2 spans from a starting point of about position 78 of SEQ ID NO:4 to an ending point of about position 153 of SEQ ID NO:4.

The enzyme Abn4 is encoded by the nucleic acid sequence represented herein as SEQ ID NO:5 and the cDNA sequence represented herein as SEQ ID NO:11. The Abn4 nucleic acid sequence encodes a 320 amino acid sequence, represented herein as SEQ ID NO:6. The signal peptide for Abn4 is located from positions 1 to about position 19 of SEQ ID NO:6, with the mature protein spanning from about position 20 to position 320 of SEQ ID NO:6. Within Abn4 is a catalytic domain (CD). The amino acid sequence containing the CD of Abn4 spans from a starting point of about position 22 of SEQ ID NO:6 to an ending point of about position 318 of SEQ ID NO:6.

The enzyme Abf3 is encoded by the nucleic acid sequence represented herein as SEQ ID NO:7 and the cDNA sequence represented herein as SEQ ID NO:12. The Abf3 nucleic acid sequence encodes a 654 amino acid sequence, represented herein as SEQ ID NO:8. The signal peptide for Abf3 is located from positions 1 to about position 18 of SEQ ID NO:8, with the mature protein spanning from about position 19 to position 654 of SEQ ID NO:8. Within Abf3 is a catalytic domain (CD). The amino acid sequence containing the CD of Abf3 spans from a starting point of about position 53 of SEQ ID NO:8 to an ending point of about position 645 of SEQ ID NO:8.

The enzymes may be isolated from a filamentous fungus. Among the preferred genera of filamentous fungi are Chrysosporium, Thielavia, Neurospora, Aureobasidium, Filibasidium, Piromyces, Cryplococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, and anamorphs and teleomorphs thereof. More preferred are Chrysosporium, Myceliophthora, Trichoderma, Aspergillus, and Fusarium. The genus and species of fungi can be defined by morphology consistent with that disclosed in Barnett and Hunter, Illustrated Genera of Imperfect Fungi, 3rd Edition, 1972, Burgess Publishing Company.

In a preferred embodiment, the fungus may be the fungal strain C1 (Accession No. VKM F-3500-D). This strain was isolated from samples of forest alkaline soil from Sola Lake, Far East of the Russian Federation and was deposited at the All-Russian Collection of Microorganisms of Russian Academy of Sciences (VKM), Bakhurhina St. 8, Moscow, Russia, 113184, under the terms of the Budapest Treaty on the International Regulation of the Deposit of Microorganisms for the Purposes of Patent Procedure on Aug. 29, 1996, as Chrysosporium lucknowense Garg 27K, VKM-F 3500 D. Various mutant strains of C1 have been produced and these strains have also been deposited at the All-Russian Collection of Microorganisms of Russian Academy of Sciences (VKM), Bakhurhina St. 8, Moscow, Russia, 113184, under the terms of the Budapest Treaty on the International Regulation of the Deposit of Microorganisms for the Purposes of Patent Procedure on Sep. 2, 1998 or at the Centraal Bureau voor Schimmelcultures (CBS), Uppsalalaan 8, 3584 CT Utrecht, The Netherlands for the purposes of Patent Procedure on Dec. 5, 2007. For example, Strain C1 was mutagenised by subjecting it to ultraviolet light to generate strain UV13-6 (Accession No. VKM F-3632 D). This strain was subsequently further mutated with N-methyl-N′-nitro-N-nitrosoguanidine to generate strain NG7C-19 (Accession No. VKM F-3633 D). This latter strain in turn was subjected to mutation by ultraviolet light, resulting in strain UV18-25 (Accession No. VKM F-3631 D). This strain in turn was again subjected to mutation by ultraviolet light, resulting in strain W1L (Accession No. CBS122189), which was subsequently subjected to mutation by ultraviolet light, resulting in strain W1L#100L (Accession No. CBS122190). Strain C1 was classified as a Chrysosporium lucknowense based on morphological and growth characteristics of the microorganism, as discussed in detail in U.S. Pat. No. 6,015,707 and U.S. Pat. No. 6,573,086. Subsequently, strain C1 has been reclassified as M. thermophilia based on genetic tests. The methods of the invention, in some embodiments, may employ derivatives or mutants of the strain C1, obtained by a combination of irradiation and chemically-induced mutagenesis. The C1 strain was subsequently reclassified as Myceliophthora thermophila based on genetic tests. C. lucknowense has also appeared in the literature as Sporotrichum thermophile.

Abn1, which acts as an endo-arabinanase, cleaves the linear α-(1,5)-linked arabinan backbone. Abn2, which acts as exo-arabinanase, degrades the linear arabinan with arabinobiose as end product. Both Abn1 and Abn2 act on linear arabinose backbone, and have poor reactivity toward the arabinose side chains. Abn4, which acts as an arabinofuranosidase, does not act on the linear arabinose backbone but is able to degrade the side chains of the arabinan enabling Abn1 and Abn2 to work on the remaining ‘debranched’ arabinan (Kühnel, 2009). A composition comprising Abn1, Abn2 and Abn4 releases mainly arabinose, arabinobiose, as well as small amounts of branched oligomeric arabinan structures (Kühnel, 2009). Abf3, which acts as an arabinoxylan arabinofuranohydrolase, is also able to hydrolyze all oligomers of arabinan, including branched oligomers, into arabinose monomers.

As described herein, all four enzymes exhibited a broad pH and temperature stability in the neutral range. Thus, the multi-enzyme composition described herein is suitable for many biotechnical applications. Particularly, given that Abn1, Abn2, Abn4 and Abf3 are active at the pH optimum of typical yeasts, a multi-enzyme composition comprising these enzymes would be highly useful in the liquefaction and saccharification of sugar beet pulp for downstream bioethanol production. The multi-enzyme composition described herein is also useful for the treatment of fruits and berries in juice and wine manufacturing for more effective juice pressing and clarification to provide higher juice yields and clearer juices. The multi-enzyme compositions described herein are also useful for the production of prebiotics.

In one embodiment, the multi-enzyme composition of the present invention further includes other pectinases, hemicellulases and/or cellulases. Since, sugar beet pulp, in addition to pectin, also contains hemicellulose and cellulose, such enriched composition would be highly effective in bioethanol production processes that utilize sugar beet pulp and other pectin rich plant biomass as feedstock. Since the main polysaccharide components in fruits and berries, are pectin, hemicellulose and cellulose, such enriched multi-enzyme composition would also be highly effective in juice and wine manufacturing. Examples of suitable pectinases include, without limitation, endo-polygalacturonase, pectin/pectate lyases, pectin methyl esterases. Examples of the hemicellulases include, without limitation, xylanases, β-xylosidases, ferulic acid esterases. Examples of the cellulases include, without limitation, endo-glucanases, cellobiohydrolases, β-glucosidases. In preferred embodiments, the pectinases, hemicellulases and cellulases are isolated from the filamentous fungi listed above.

For example, in one embodiment for the production of biofuels, cellulases, pectinases, and arabinases (including arabinofuranosidases, acetyl esterase, etc.) may be used. In another embodiment for the clarification of juice, only pectinases and arabinases are needed. In another embodiment, for juice pressing cellulases are also needed in addition to the pectinases/arabinases.

The multi-enzyme compositions of the present invention may be produced using any techniques known in the art. For example, the multi-enzyme compositions may be produced using recombinant DNA technology. In one embodiment, the genes encoding the enzymes described herein are introduced in a host cell so that the resultant genetically modified host cell is capable of expressing the genes and producing the multi-enzyme composition described above. In a further embodiment, the genes encoding the enzymes described herein may be introduced in a host cell that also contains genes encoding the other pectinases, cellulases and/or hemi-cellulases described above so that the resultant genetically modified host cell is capable of expressing the genes and producing the enriched multi-enzyme composition described above. A number of methods for introducing genes in host cells are known in the art and are included in the present invention. In some embodiments, the host cell is a fungal cell. In one embodiment, the host cell is a C1 fungal cell.

Further, described herein is the characterization of the novel oligomeric arabinan structures formed by enzymatic degradation of sugar beet arabinan with a composition comprising Abn1, Abn2 and Abn4. The resultant oligomers were separated by fractionation based on size and were characterized using NMR analysis. Two main series of branched arabinan oligosaccharides were identified, both having an α-(1,5)-linked arabinan backbone. One series was found to contain only single substituted α-(1,3)-linked arabinose(s) attached to the backbone, the other series consisted of a double substituted α-(1,2,3,5)-linked arabinan structure within the molecule. This is believed to be the first report of isolation and purification of branched arabinan oligosaccharides containing α-(1,2)-, α-(1,3)- or α-(1,2,3)-structures that differ from linear α-(1,5)-arabinan oligosaccharides.

The branched arabinan oligomers can be used as prebiotics. Prebiotics are non-digestible foods that stimulate the growth and/or activity of bacteria in the digestive system which are beneficial to the health of the body. Prebiotic oligosaccharides are increasingly added to foods for their health benefits. It is expected that the branched arabinan oligomers will work as effective prebiotics, since they are expected to penetrate the gut further where they might influence the microbial composition in the more distal parts of the colon (Voragen, Technological aspects of functional food-related carbohydrates, Trends in Food Science and Technology, 9:328-335, 1998).

Accordingly, in one embodiment, the present invention includes a method for hydrolyzing arabinans present in a plant biomass, comprising contacting the plant biomass with a multi-enzyme composition, wherein the multi-enzyme composition comprises the enzymes Abn1 (SEQ ID NO:2), Abn4 (SEQ ID NO:6) and Abf3 (SEQ ID NO:8). In another embodiment, the multi-enzyme composition further comprises Abn2 (SEQ ID NO:4). In various embodiments, the multi-enzyme composition is able to degrade at least about 50%, at least about 55%, at least about 60%, at least about 65%, at least about 70%, at least about 75%, at least about 80%, at least about 85%, at least about 90%, at least about 95%, or at least about 99% of the arabinan present in the plant biomass to arabinose.

In another embodiment, the present invention includes a multi-enzyme composition comprising the enzymes Abn1 (SEQ ID NO:2), Abn2 (SEQ ID NO:4), Abn4 (SEQ ID NO:6) and Abf3 (SEQ ID NO:8) wherein the multi-enzyme composition is able to degrade at least about 50%, at least about 55%, at least about 60%, at least about 65%, at least about 70%, at least about 75%, at least about 80%, at least about 85%, at least about 90%, at least about 95%, or at least about 99% of the arabinan present in a plant biomass to arabinose.

The plant biomass may be derived from a number of plant sources, examples of which include, without limitation, sugar beet, soybeans, olives, apples, and black currants. In a preferred embodiment, the plant biomass may be derived from sugar beet. In some embodiments, the specific activity of Abn1 towards linear arabinan may be 5-40, 10-35, or 20-30 U/mg, specific activity of Abn2 towards linear arabinan may be 1-20, 2-18, 3-15, or 8-11 U/mg, and specific activity of Abn4 towards branched arabinan may be 1-20, 2-15, or 5-12 U/mg. In some embodiments, the specific activity of Abf3 towards p-Nitrophenyl-α-arabinofuranose may be 5-45, 10-40, 15-35, or 20-30 U/mg. In some embodiments, the specific activity of Abn1 towards linear arabinan may be 26 U/mg, specific activity of Abn2 towards linear arabinan may be 7.1 U/mg, and specific activity of Abn4 towards branched arabinan may be 9.5 U/mg. In some embodiments, the specific activity of Abf3 towards p-Nitrophenyl-α-arabinofuranose may be 28.4 U/mg.

In another embodiment, the present invention includes a method to prepare a prebiotic. The method comprises contacting a plant biomass comprising arabinans with a multi-enzyme composition that includes Abn1, Abn2, and Abn4. The multi-enzyme complex is capable of degrading the arabinans in the plant biomass into branched arabinan oligomers. In a further embodiment, the present invention includes a multi-enzyme composition that is useful in the preparation of prebiotics comprising the enzymes Abn1, Abn2, and Abn4, wherein the multi-enzyme composition is able to hydrolyze arabinan present in the plant biomass into branched arabinan oligomers. In another embodiment, the present invention includes a prebiotic that includes branched arabinan oligomers. In another embodiment, the enzymes are dosed in a ratio of 1:1:1. In another embodiment, the Abn1:Abn2:Abn4 are dosed in a ratio of 1-10:1:1-5. In another embodiment, Abn1, Abn2, and Abn4 are added to a cellulase mixture in a ratio of cellulase mixture:Abn1:Abn2:Abn4 is 10-50:1-10:1:1-5. In one embodiment, a 10 mg/g cellulase mixture is used and 1 mg/g of each pure enzyme is added.

The structure of the branched arabinan oligomers includes a α-(1,5)-linked arabinan backbone, and a) single substituted α-(1,3)-linked arabinose monomers attached to the backbone, or b) double substituted α-(1,2,3,5)-linked arabinose monomers attached to the backbone, c) or both. The plant biomass may be derived from a number of plant sources, examples of which include, without limitation, sugar beet, soybeans, olives, apples, and black currants.

In further embodiments, the present invention includes a method to prepare a fruit juice or wine from a plant biomass (such as fruits or berries) or a method for saccharification of a plant biomass. The method comprises contacting a plant biomass comprising arabinans with a multi-enzyme composition, wherein the multi-enzyme composition comprises Abn1, Abn2, and Abn4, and Abf3. The multi-enzyme complex is capable of degrading the arabinans in the plant biomass into linear and branched arabinanose oligomers. The plant biomass may contain pectins, hemi-celluloses and/or celluloses. In some embodiments, the multi-enzyme composition may further comprise one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase and wherein the plant biomass comprises pectins, hemi-celluloses and/or celluloses. In another embodiment, the enzymes are dosed in a ratio of 1:1:1:1. In another embodiment, the Abn1:Abn2:Abn4:Abf3 are dosed in a ratio of 1-10:1:1-5:1-5. In another embodiment, Abn1, Abn2, Abn4, and Abf3 are added to a cellulase mixture in a ratio of cellulase mixture:Abn1:Abn2:Abn4:Abf3 is 10-50:1-10:1:1-5:1-5. In one embodiment, a 10 mg/g cellulase mixture is used and 1 mg/g of each pure enzyme is added.

In another embodiment, the present invention includes a recombinant micro-organism that is genetically modified to express Abn1, Abn2, Abn4 and Abf3. In some embodiments, the recombinant microorganism may express one or more of the following additional enzymes: endo-polygalacturonase, pectin/pectate lyase, pectin methyl esterase, endo-glucanase, cellobiohydrolase, β-glucosidase, xylanase, β-xylosidase and ferulic acid esterase and wherein the plant biomass comprises pectins, hemi-celluloses and/or celluloses. The additional enzymes may be endogenously expressed or the microorganism may be genetically modified to express them. In some embodiments, the microorganism is a filamentous fungus.

Proteins of the present invention, at least one protein of the present invention, compositions comprising such protein(s) of the present invention, and multi-enzyme compositions (examples of which are described above) may be used in any method where it is desirable to hydrolyze glycosidic linkages in lignocellulosic material, or any other method wherein enzymes of the same or similar function are useful.

In some embodiments, the methods may be performed one or more times in whole or in part. That is, one may perform one or more pretreatments, followed by one or more reactions with a protein of the present invention, composition or product of the present invention and/or accessory enzyme. The enzymes may be added in a single dose, or may be added in a series of small doses. Further, the entire process may be repeated one or more times as necessary. Therefore, one or more additional treatments with heat and enzymes are contemplated.

The present invention also provides enzyme combinations that can be used to break down lignocellulose material. Such enzyme combinations or mixtures can include a multi-enzyme composition that contains at least one protein of the present invention in combination with one or more additional proteins of the present invention or one or more enzymes or other proteins from other microorganisms, plants, or similar organisms. Synergistic enzyme combinations and related methods are contemplated. In particular, the enzymes of the present invention act in the multi-enzyme composition to aid in the delignify of the lignocellulose material. The invention includes methods to identify the optimum ratios and compositions of enzymes with which to degrade each lignocellulosic material. These methods entail tests to identify the optimum enzyme composition and ratios for efficient conversion of any lignocellulosic substrate to its constituent sugars. The Examples below include assays that may be used to identify optimum ratios and compositions of enzymes with which to degrade lignocellulosic materials.

Any combination of the proteins disclosed herein is suitable for use in the multi-enzyme compositions of the present invention. Due to the complex nature of most biomass sources, which can contain cellulose, hemicellulose, pectin, lignin, protein, and ash, among other components, preferred enzyme combinations may contain enzymes with a range of substrate specificities that work together to degrade biomass in the most efficient manner. One example of a multi-enzyme complex for lignocellulose saccharification is a mixture of cellobiohydrolase(s), xylanase(s), endoglucanase(s), β-glucosidase(s), β-xylosidase(s), peptidase(s), and accessory enzymes. However, it is to be understood that any of the enzymes described specifically herein can be combined with any one or more of the enzymes described herein or with any other available and suitable enzymes, to produce a multi-enzyme composition. The invention is not restricted or limited to the specific exemplary combinations listed below.

The enzymes of the multi-enzyme composition can be provided by a variety of sources. In one embodiment, the enzymes can be produced by growing organisms such as bacteria, algae, fungi, and plants which produce the enzymes naturally or by virtue of being genetically modified to express the enzyme or enzymes. In another embodiment, at least one enzyme of the multi-enzyme composition is a commercially available enzyme.

In some embodiments, the multi-enzyme compositions comprise an accessory enzyme. An accessory enzyme can have the same or similar function or a different function as an enzyme or enzymes in the core set of enzymes. These enzymes have been described elsewhere herein, and can generally include peptidases, cellulases, xylanases, ligninases, amylases, lipidases, or glucuronidases, for example. An accessory enzyme or enzyme mix may be composed of enzymes from (1) commercial suppliers; (2) cloned genes expressing enzymes; (3) complex broth (such as that resulting from growth of a microbial strain in media, wherein the strains secrete proteins and enzymes into the media); (4) cell lysates of strains grown as in (3); and, (5) plant material expressing enzymes.

The multi-enzyme compositions, in some embodiments, comprise a biomass comprising microorganisms or a crude fermentation product of microorganisms. A crude fermentation product refers to the fermentation broth which has been separated from the microorganism biomass (by filtration, for example). In general, the microorganisms are grown in fermentors, optionally centrifuged or filtered to remove biomass, and optionally concentrated, formulated, and dried to produce an enzyme(s) or a multi-enzyme composition that is a crude fermentation product. In other embodiments, enzyme(s) or multi-enzyme compositions produced by the microorganism (including a genetically modified microorganism as described below) are subjected to one or more purification steps, such as ammonium sulfate precipitation, chromatography, and/or ultrafiltration, which result in a partially purified or purified enzyme(s). If the microorganism has been genetically modified to express the enzyme(s), the enzyme(s) will include recombinant enzymes. If the genetically modified microorganism also naturally expresses the enzyme(s) or other enzymes useful for the degradation of protein, the enzyme(s) may include both naturally occurring and recombinant enzymes.

Another embodiment of the present invention relates to a composition comprising at least about 500 ng, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an isolated protein comprising any of the proteins or homologues, variants, or fragments thereof discussed herein. Such a composition of the present invention may include any carrier with which the protein is associated by virtue of the protein preparation method, a protein purification method, or a preparation of the protein for use in any method according to the present invention. For example, such a carrier can include any suitable buffer, extract, or medium that is suitable for combining with the protein of the present invention so that the protein can be used in any method described herein according to the present invention.

In some embodiments, the present invention comprises a kit comprising at least one oligonucleotide of the present invention.

In some embodiments, the present invention comprises methods for producing a protein of the present invention, comprising culturing a cell that has been transfected with a nucleic acid molecule comprising a nucleic acid sequence encoding the protein, and expressing the protein with the transfected cell. In some embodiments, the present invention further comprises recovering the protein from the cell or from a culture comprising the cell.

In some embodiments, the genetically modified organism is a plant, alga, fungus or bacterium. In some embodiments, the fungus is yeast, mushroom or filamentous fungus.

In some embodiments, the algae is selected from the group consisting of: Chlorophyta, Rhodophyta, Glaucophyta, Chlorarchniophytes, Eugleinds, Heterokonts, Bacillariophyceae, Axodine, Bolidomonas, Eustigmatophyceae, Phaeophyceae, Chrysophyceae, Raphidophyceae, Synurophyceae, Xanthophycease, Cryptophyta, Dinoflagellates, Haptophyta, Chlorella, Dunaliella, Haematococcus, Volvox, Synechocystis. Phaedactylum tricornatum, Protococus and Pleurococus.

In some embodiments, the filamentous fungus is from a genus selected from the group consisting of: Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Talaromyces, Gibberella, Myceliophthora, Mucor, Aspergillus, Fusarium, Humicola, and Trichoderma. In some embodiments, the filamentous fungus is selected from the group consisting of: Trichoderma reesei, Chrysosporium lucknowense, Aspergillus japonicus, Aspergillus niger Penicillium canescens, Penicillium solitum, Penicillium funiculosum, Talaromyces emersonii and Talaromyces flavus.

In some embodiments, the genetically modified organism has been genetically modified to express at least one additional enzyme. In some embodiments, the additional enzyme is an enzyme selected from the group consisting of: cellulase, glucosidase, xylanase, xylosidase, ligninase, glucuronidase, arabinofuranosidase, arabinase, arabinogalactanase, esterase, lipase, pectinase, glucomannanase, amylase, laminarinase, xyloglucanase, galactanase, galactosidase, glucoamylase, pectate and pectin lyase, chitosanases, exo-β-D-glucosaminidase, and cellobiose dehydrogenase.

In one embodiment of the invention, one or more enzymes of the invention is bound to a solid support, i.e., an immobilized enzyme. As used herein, an immobilized enzyme includes immobilized isolated enzymes, immobilized microbial cells which contain one or more enzymes of the invention, other stabilized intact cells that produce one or more enzymes of the invention, and stabilized cell/membrane homogenates. Stabilized intact cells and stabilized cell/membrane homogenates include cells and homogenates from naturally occurring microorganisms expressing the enzymes of the invention and preferably, from genetically modified microorganisms as disclosed elsewhere herein. Thus, although methods for immobilizing enzymes are discussed below, it will be appreciated that such methods are equally applicable to immobilizing microbial cells and in such an embodiment, the cells can be lysed, if desired.

A variety of methods for immobilizing an enzyme are disclosed in Industrial Enzymology 2nd Ed., Godfrey, T. and West, S. Eds., Stockton Press, New York, N.Y., 1996, pp. 267-272; Immobilized Enzymes, Chibata, I. Ed., Halsted Press, New York, N.Y., 1978; Enzymes and Immobilized Cells in Biotechnology, Laskin, A. Ed., Benjamin/Cummings Publishing Co., Inc., Menlo Park, Calif., 1985; and Applied Biochemistry and Bioengineering, Vol. 4, Chibata, I. and Wingard, Jr., L. Eds, Academic Press, New York, N.Y., 1983.

Briefly, a solid support refers to any solid organic, biopolymer or inorganic supports that can form a bond with an enzyme without significantly effecting the activity of the enzyme. Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates. Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand. In one embodiment, the solid support is selected from the group consisting of stabilized intact cells and/or crude cell homogenates (e.g., produced from the microbial host cells expressing recombinant enzymes, alone or in combination with natural enzymes). Preparation of such supports requires a minimum of handling and cost. Additionally, such supports provide excellent stability of the enzyme.

Stabilized intact cells and/or cell/membrane homogenates can be produced, for example, by using bifunctional crosslinkers (e.g., glutaraldehyde) to stabilize cells and cell homogenates. In both the intact cells and the cell membranes, the cell wall and membranes act as immobilizing supports. In such a system, integral membrane proteins are in the “best” lipid membrane environment. Whether starting with intact cells or homogenates, in this system the cells are either no longer “alive” or “metabolizing”, or alternatively, are “resting” (i.e., the cells maintain metabolic potential and active enzyme, but under the culture conditions are not growing); in either case, the immobilized cells or membranes serve as biocatalysts.

An enzyme of the invention can be bound to a solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports in a bead form are particularly well-suited. The particle size of an adsorption solid support can be selected such that the immobilized enzyme is retained in the reactor by a mesh filter while the substrate is allowed to flow through the reactor at a desired rate. With porous particulate supports it is possible to control the adsorption process to allow enzymes or cells to be embedded within the cavity of the particle, thus providing protection without an unacceptable loss of activity.

Cross-linking of an enzyme to a solid support involves forming a chemical bond between a solid support and the enzyme. It will be appreciated that although cross-linking generally involves linking the enzyme to a solid support using an intermediary compound, it is also possible to achieve a covalent bonding between the enzyme and the solid support directly without the use of an intermediary compound. Cross-linking commonly uses a bifunctional or multifunctional reagent to activate and attach a carboxyl group, amino group, sulfur group, hydroxy group or other functional group of the enzyme to the solid support. The term “activate” refers to a chemical transformation of a functional group which allows a formation of a bond at the functional group. Exemplary amino group activating reagents include water-soluble carbodiimides, glutaraldehyde, cyanogen bromide, N-hydroxysuccinimide esters, triazines, cyanuric chloride, and carbonyl diimidazole. Exemplary carboxyl group activating reagents include water-soluble carbodiimides, and N-ethyl-5-phenylisoxazolium-3-sulfonate. Exemplary tyrosyl group activating reagents include diazonium compounds. And exemplary sulfhydryl group activating reagents include dithiobis-5,5′-(2-nitrobenzoic acid), and glutathione-2-pyridyl disulfide. Systems for covalently linking an enzyme directly to a solid support include Eupergit®, a polymethacrylate bead support available from Rohm Pharma (Darmstadt, Germany), kieselguhl (Macrosorbs), available from Sterling Organics, kaolinite available from English China Clay as “Biofix” supports, silica gels which can be activated by silanization, available from W. R. Grace, and high-density alumina, available from UOP (Des Plains, Ill.).

Entrapment can also be used to immobilize an enzyme. Entrapment of an enzyme involves formation of inter alia, gels (using organic or biological polymers), vesicles (including microencapsulation), semipermeable membranes or other matrices. Exemplary materials used for entrapment of an enzyme include collagen, gelatin, agar, cellulose triacetate, alginate, polyacrylamide, polystyrene, polyurethane, epoxy resins, carrageenan, and egg albumin. Some of the polymers, in particular cellulose triacetate, can be used to entrap the enzyme as they are spun into a fiber. Other materials such as polyacrylamide gels can be polymerized in solution to entrap the enzyme. Still other materials such as polyglycol oligomers that are functionalized with polymerizable vinyl end groups can entrap enzymes by forming a cross-linked polymer with UV light illumination in the presence of a photosensitizer.

Proteins of the present invention, at least one protein of the present invention, compositions comprising such protein(s) of the present invention, and multi-enzyme compositions (examples of which are described above) may be used in any method where it is desirable to degrade protein, or any other method wherein enzymes of the same or similar function are useful.

In one embodiment, the present invention includes the use of at least one protein of the present invention, compositions comprising at least one protein of the present invention, or multi-enzyme compositions in methods for hydrolyzing protein therefrom. In one embodiment, the method comprises contacting the protein with an effective amount of one or more proteins of the present invention, composition comprising at least one protein of the present invention, or a multi-enzyme composition, whereby at least one amino acid is liberated.

Typically, the amount of enzyme or enzyme composition contacted with the protein will depend upon the amount of the protein, order of the sequence, or environmental conditions. In some embodiments, the amount of enzyme or enzyme composition contacted with the protein may be from about 0.1 to about 200 mg enzyme or enzyme composition per gram of protein; in other embodiments, from about 3 to about 20 mg enzyme or enzyme composition per gram of protein. The invention encompasses the use of any suitable or sufficient amount of enzyme or enzyme composition between about 0.1 mg and about 200 mg enzyme per gram protein, in increments of 0.05 mg (i.e., 0.1 mg, 0.15 mg, 0.2 mg . . . 199.9 mg, 199.95 mg, 200 mg).

In some embodiments, the present invention provides methods for improving the nutritional quality of food (or animal feed) comprising adding to the food (or the animal feed) at least one protein of the present invention. In some embodiments, the present invention provides methods for improving the nutritional quality of the food (or animal feed) comprising pretreating the food (or the animal feed) with at least one isolated protein of the present invention. In some embodiments, the proteins of the present invention may be used as part of nutritional supplements. In some embodiments, the proteins of the present invention may be used as part of digestive aids, and may help in providing relief from digestive disorders such as acid reflux and celiac disease.

Nucleic Acid and Amino Acid

As used herein, reference to an isolated protein or polypeptide in the present invention, including any of the enzymes disclosed herein, includes full-length proteins and their glycosylated or otherwise modified forms, fusion proteins, or any fragment or homologue or variant of such a protein. More specifically, an isolated protein, such as an enzyme according to the present invention, is a protein (including a polypeptide or peptide) that has been removed from its natural milieu (i.e., that has been subject to human manipulation) and can include purified proteins, partially purified proteins, recombinantly produced proteins, synthetically produced proteins, proteins complexed with lipids, soluble proteins, and isolated proteins associated with other proteins, for example. As such, “isolated” does not reflect the extent to which the protein has been purified. Preferably, an isolated protein of the present invention is produced recombinantly. In addition, and by way of example, a “C. lucknowense protein” or “C. lucknowense enzyme” refers to a protein (generally including a homologue or variant of a naturally occurring protein) from Chrysosporium lucknowense or to a protein that has been otherwise produced from the knowledge of the structure (e.g., sequence) and perhaps the function of a naturally occurring protein from Chrysosporium lucknowense. In other words, a C. lucknowense protein includes any protein that has substantially similar structure and function of a naturally occurring C. lucknowense protein or that is a biologically active (i.e., has biological activity) homologue or variant of a naturally occurring protein from C. lucknowense as described in detail herein. As such, a C. lucknowense protein can include purified, partially purified, recombinant, mutated/modified and synthetic proteins.

According to the present invention, the terms “modification,” “mutation,” and “variant” can be used interchangeably, particularly with regard to the modifications/mutations to the amino acid sequence of a C. lucknowense protein (or nucleic acid sequences) described herein. An isolated protein according to the present invention can be isolated from its natural source, produced recombinantly or produced synthetically.

According to the present invention, the terms “modification” and “mutation” can be used interchangeably, particularly with regard to the modifications/mutations to the primary amino acid sequences of a protein or peptide (or nucleic acid sequences) described herein. The term “modification” can also be used to describe post-translational modifications to a protein or peptide including, but not limited to, methylation, farnesylation, carboxymethylation, geranyl geranylation, glycosylation, phosphorylation, acetylation, myristoylation, prenylation, palmitation, and/or amidation. Modification can also include the cleavage of a signal peptide, or methionine, or other portions of the peptide that require cleavage to generate the mature peptide. Modifications can also include, for example, complexing a protein or peptide with another compound. Such modifications can be considered to be mutations, for example, if the modification is different than the post-translational modification that occurs in the natural, wild-type protein or peptide.

As used herein, the terms “homologue” or “variants” are used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide), insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue or variant can have either enhanced, decreased, or substantially similar properties as compared to the naturally occurring protein or peptide. A homologue or variant can include an agonist of a protein or an antagonist of a protein.

Homologues or variants can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Homologous can also be the result of a gene duplication and rearrangement, resulting in a different location. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues or variants can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in protein homologues or variants, as compared to the wild-type protein, either agonize, antagonize, or do not substantially change, the basic biological activity of the homologue or variant as compared to the naturally occurring protein. Modifications of a protein, such as in a homologue or variant, may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

According to the present invention, an isolated protein, including a biologically active homologue, variant, or fragment thereof, has at least one characteristic of biological activity of a wild-type, or naturally occurring, protein. As discussed above, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). The biological activity of a protein of the present invention can include an enzyme activity (catalytic activity and/or substrate binding activity), endopeptidase, exopeptidase, metallopeptidase, amino peptidase, carboxy peptidase, amino acid-specific peptidase or any other activity disclosed herein. Specific biological activities of the proteins disclosed herein are described in detail above and in the Examples. Methods of detecting and measuring the biological activity of a protein of the invention include, but are not limited to, the assays described in the Examples section below. Such assays include, but are not limited to, measurement of enzyme activity (e.g., catalytic activity), measurement of substrate binding, and the like. It is noted that an isolated protein of the present invention (including homologues or variants) is not required to have a biological activity such as catalytic activity. A protein can be a truncated, mutated or inactive protein, or lack at least one activity of the wild-type enzyme, for example. Inactive proteins may be useful in some screening assays, for example, or for other purposes such as antibody production.

Methods to measure protein expression levels of a protein according to the invention include, but are not limited to: western blotting, immunocytochemistry, flow cytometry or other immunologic-based assays; assays based on a property of the protein including but not limited to, ligand binding or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of one protein to another include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR).

Many of the enzymes and proteins of the present invention may be desirable targets for modification and use in the processes described herein. These proteins have been described in terms of function and amino acid sequence (and nucleic acid sequence encoding the same) of representative wild-type proteins. In one embodiment of the invention, homologues or variants of a given protein (which can include related proteins from other organisms or modified forms of the given protein) are encompassed for use in the invention. Homologues or variants of a protein encompassed by the present invention can comprise, consist essentially of, or consist of, in one embodiment, an amino acid or nucleic acid sequence that is at least about 35% identical, at least about 40% identical, at least about 45% identical, at least about 50% identical, at least about 55% identical, at least about 60% identical, at least about 65% identical, at least about 70% identical, at least about 75% identical, at least about 80% identical, at least about 85% identical, at least about 90% identical, at least about 95% identical, at least about 96% identical, at least about 97% identical, at least about 98% identical, at least about 99% identical, or any percent identity between 35% and 99%, in whole integers (i.e., 36%, 37%, etc.), to an amino acid or nucleic acid sequence disclosed herein that represents the amino acid or nucleic acid sequence of an enzyme or protein according to the invention (including a biologically active domain of a full-length protein). Preferably, the amino acid or nucleic acid sequence of the homologue or variant has a biological activity of the wild-type or reference protein or of a biologically active domain thereof (e.g., a catalytic domain). When denoting mutation positions, the amino acid position of the wild-type is typically used. The wild-type can also be referred to as the “parent.” Additionally, any generation before the variant at issue can be a parent.

In one embodiment, a protein of the present invention comprises, consists essentially of, or consists of an amino acid or nucleic acid sequence that, alone or in combination with other characteristics of such proteins disclosed herein, is less than 100% identical to nucleic acid sequence from SEQ ID NO: 1, 3, 5, or 7 or an amino acid sequence selected from Sequences SEQ ID NO: 2, 4, 6, or 8 (i.e., a homologue or variant). For example, a protein of the present invention can be less than 100% identical, in combination with being at least about 35% identical, to a given disclosed sequence. In another aspect of the invention, a homologue or variant according to the present invention has an amino acid or nucleic acid sequence that is less than about 99% identical to any of such amino acid or nucleic acid sequences, and in another embodiment, is less than about 98% identical to any of such amino acid sequences, and in another embodiment, is less than about 97% identical to any of such amino acid or nucleic acid sequences, and in another embodiment, is less than about 96% identical to any of such amino acid or nucleic acid sequences, and in another embodiment, is less than about 95% identical to any of such amino acid or nucleic acid sequences, and in another embodiment, is less than about 94% identical to any of such amino acid or nucleic acid sequences, and in another embodiment, is less than about 93% identical to any of such amino acid or nucleic acid sequences, and in another embodiment, is less than about 92% identical to any of such amino acid or nucleic acid sequences, and in another embodiment, is less than about 91% identical to any of such amino acid or nucleic acid sequences, and in another embodiment, is less than about 90% identical to any of such amino acid or nucleic acid sequences, and so on, in increments of whole integers.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); (2) a BLAST 2 alignment (using the parameters described below); (3) PSI-BLAST with the standard default parameters (Position-Specific Iterated BLAST; and/or (4) CAZy homology determined using standard default parameters from the Carbohydrate Active EnZymes database (Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12).

It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues or variants. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

A protein of the present invention can also include proteins having an amino acid sequence comprising at least 10 contiguous amino acid residues of any of the sequences described herein (i.e., 10 contiguous amino acid residues having 100% identity with 10 contiguous amino acids of the amino acid sequences of Sequences SEQ ID NO: 2, 4, 6, and 8). In other embodiments, a homologue or variant of a protein amino acid sequence includes amino acid sequences comprising at least 20, or at least 30, or at least 40, or at least 50, or at least 75, or at least 100, or at least 125, or at least 150, or at least 175, or at least 150, or at least 200, or at least 250, or at least 300, or at least 350 contiguous amino acid residues of any of the amino acid sequence represented disclosed herein. Even small fragments of proteins without biological activity are useful in the present invention, for example, in the preparation of antibodies against the full-length protein or in a screening assay (e.g., a binding assay). Fragments can also be used to construct fusion proteins, for example, where the fusion protein comprises functional domains from two or more different proteins (e.g., a CBM from one protein linked to a CD from another protein). In one embodiment, a homologue or variant has a measurable or detectable biological activity associated with the wild-type protein (e.g., enzymatic activity).

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, a protein of the present invention, including a homologue or variant, includes a protein having an amino acid sequence that is sufficiently similar to a natural amino acid sequence that a nucleic acid sequence encoding the homologue or variant is capable of hybridizing under moderate, high or very high stringency conditions (described below) to (i.e., with) a nucleic acid molecule encoding the natural protein (i.e., to the complement of the nucleic acid strand encoding the natural amino acid sequence). Preferably, a homologue or variant of a protein of the present invention is encoded by a nucleic acid molecule comprising a nucleic acid sequence that hybridizes under low, moderate, or high stringency conditions to the complement of a nucleic acid sequence that encodes a protein comprising, consisting essentially of, or consisting of an amino acid sequence represented by any of SEQ ID NO: 2, 4, 6, or 8. Such hybridization conditions are described in detail below.

A nucleic acid sequence complement of nucleic acid sequence encoding a protein of the present invention refers to the nucleic acid sequence of the nucleic acid strand that is complementary to the strand which encodes the protein. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence such as the amino acid sequences of SEQ ID NO: 2, 4, 6, or 8, and/or with the complement of the nucleic acid sequence that encodes an amino acid sequence such as the amino acid sequences of SEQ ID NO: 2, 4, 6, or 8. Methods to deduce a complementary sequence are known to those skilled in the art. It should be noted that since nucleic acid sequencing technologies are not entirely error-free, the sequences presented herein, at best, represent apparent sequences of the proteins of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% or 95% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

The minimum size of a protein and/or homologue or variant of the present invention is a size sufficient to have biological activity or, when the protein is not required to have such activity, sufficient to be useful for another purpose associated with a protein of the present invention, such as for the production of antibodies that bind to a naturally occurring protein. In one embodiment, the protein of the present invention is at least 20 amino acids in length, or at least about 25 amino acids in length, or at least about 30 amino acids in length, or at least about 40 amino acids in length, or at least about 50 amino acids in length, or at least about 60 amino acids in length, or at least about 70 amino acids in length, or at least about 80 amino acids in length, or at least about 90 amino acids in length, or at least about 100 amino acids in length, or at least about 125 amino acids in length, or at least about 150 amino acids in length, or at least about 175 amino acids in length, or at least about 200 amino acids in length, or at least about 250 amino acids in length, and so on up to a full length of each protein, and including any size in between in increments of one whole integer (one amino acid). There is no limit, other than a practical limit, on the maximum size of such a protein in that the protein can include a portion of a protein or a full-length protein, plus additional sequence (e.g., a fusion protein sequence), if desired.

The present invention also includes a fusion protein that includes a domain of a protein of the present invention (including a homologue or variant) attached to one or more fusion segments, which are typically heterologous in sequence to the protein sequence (i.e., different than protein sequence). Suitable fusion segments for use with the present invention include, but are not limited to, segments that can: enhance a protein's stability; provide other desirable biological activity; and/or assist with the purification of the protein (e.g., by affinity chromatography). A suitable fusion segment can be a domain of any size that has the desired function (e.g., imparts increased stability, solubility, action or biological activity; and/or simplifies purification of a protein). Fusion segments can be joined to amino and/or carboxyl termini of the domain of a protein of the present invention and can be susceptible to cleavage in order to enable straight-forward recovery of the protein. Fusion proteins are preferably produced by culturing a recombinant cell transfected with a fusion nucleic acid molecule that encodes a protein including the fusion segment attached to either the carboxyl and/or amino terminal end of a domain of a protein of the present invention. Accordingly, proteins of the present invention also include expression products of gene fusions (for example, used to overexpress soluble, active forms of the recombinant protein), of mutagenized genes (such as genes having codon modifications to enhance gene transcription and translation), and of truncated genes (such as genes having membrane binding modules removed to generate soluble forms of a membrane protein, or genes having signal sequences removed which are poorly tolerated in a particular recombinant host).

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived.

Further embodiments of the present invention include nucleic acid molecules that encode a protein of the present invention, as well as homologues, variants, or fragments of such nucleic acid molecules. A nucleic acid molecule of the present invention includes a nucleic acid molecule comprising, consisting essentially of, or consisting of a nucleic acid sequence encoding any of the isolated proteins disclosed herein, including a fragment or a homologue or variant of such proteins, described above. Nucleic acid molecules can include a nucleic acid sequence that encodes a fragment of a protein that does not have biological activity, and can also include portions of a gene or polynucleotide encoding the protein that are not part of the coding region for the protein (e.g., introns or regulatory regions of a gene encoding the protein). Nucleic acid molecules can include a nucleic acid sequence that is useful as a probe or primer (oligonucleotide sequences).

In one embodiment, a nucleic acid molecule of the present invention includes a nucleic acid molecule comprising, consisting essentially of, or consisting of, a nucleic acid sequence represented in SEQ ID NO: 1, 3, 5, or 7 or fragments or homologues or variants thereof. Preferably, the nucleic acid sequence encodes a protein (including fragments and homologues or variants thereof) useful in the invention, or encompasses useful oligonucleotides or complementary nucleic acid sequences.

In one embodiment, a nucleic molecule of the present invention includes a nucleic acid molecule comprising, consisting essentially of, or consisting of, a nucleic acid sequence encoding an amino acid sequence represented in SEQ ID NO: 1-8 or fragments or homologues or variants thereof. Preferably, the nucleic acid sequence encodes a protein (including fragments and homologues or variants thereof) useful in the invention, or encompasses useful oligonucleotides or complementary nucleic acid sequences.

In one embodiment, such nucleic acid molecules include isolated nucleic acid molecules that hybridize under moderate stringency conditions, and more preferably under high stringency conditions, and even more preferably under very high stringency conditions, as described above, with the complement of a nucleic acid sequence encoding a protein of the present invention (i.e., including naturally occurring allelic variants encoding a protein of the present invention). Preferably, an isolated nucleic acid molecule encoding a protein of the present invention comprises a nucleic acid sequence that hybridizes under moderate, high, or very high stringency conditions to the complement of a nucleic acid sequence that encodes a protein comprising an amino acid sequence represented in SEQ ID NO: 1-8.

In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule, and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a protein of the present invention or to form stable hybrids under stringent conditions with natural gene isolates. An isolated nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein of the present invention can vary due to degeneracies. It is noted that a nucleic acid molecule of the present invention is not required to encode a protein having protein activity. A nucleic acid molecule can encode a truncated, mutated or inactive protein, for example. In addition, nucleic acid molecules of the invention are useful as probes and primers for the identification, isolation and/or purification of other nucleic acid molecules. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

According to the present invention, reference to a gene includes all nucleic acid sequences related to a natural (i.e. wild-type) gene, such as regulatory regions that control production of the protein encoded by that gene (such as, but not limited to, transcription, translation or post-translation control regions) as well as the coding region itself. In another embodiment, a gene can be a naturally occurring allelic variant that includes a similar but not identical sequence to the nucleic acid sequence encoding a given protein. Allelic variants have been previously described above. Genes can include or exclude one or more introns or any portions thereof or any other sequences or which are not included in the cDNA for that protein. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning, etc.) or chemical synthesis. Isolated nucleic acid molecules include any nucleic acid molecules and homologues or variants thereof that are part of a gene described herein and/or that encode a protein described herein, including, but not limited to, natural allelic variants and modified nucleic acid molecules (homologues or variants) in which nucleotides have been inserted, deleted, substituted, and/or inverted in such a manner that such modifications provide the desired effect on protein biological activity or on the activity of the nucleic acid molecule. Allelic variants and protein homologues or variants (e.g., proteins encoded by nucleic acid homologues or variants) have been discussed in detail above.

A nucleic acid molecule homologue or variant (i.e., encoding a homologue or variant of a protein of the present invention) can be produced using a number of methods known to those skilled in the art (see, for example, Sambrook et al.). For example, nucleic acid molecules can be modified using a variety of techniques including, but not limited to, by classic mutagenesis and recombinant DNA techniques (e.g., site-directed mutagenesis, chemical treatment, restriction enzyme cleavage, ligation of nucleic acid fragments and/or PCR amplification), or synthesis of oligonucleotide mixtures and ligation of mixture groups to “build” a mixture of nucleic acid molecules and combinations thereof. Another method for modifying a recombinant nucleic acid molecule encoding a protein is gene shuffling (i.e., molecular breeding) (See, for example, U.S. Pat. No. 5,605,793 to Stemmer; Minshull and Stemmer; 1999, Curr. Opin. Chem. Biol. 3:284-290; Stemmer, 1994, P.N.A.S. USA 91:10747-10751). This technique can be used to efficiently introduce multiple simultaneous changes in the protein. Nucleic acid molecule homologues or variants can be selected by hybridization with a gene or polynucleotide, or by screening for the function of a protein encoded by a nucleic acid molecule (i.e., biological activity).

The minimum size of a nucleic acid molecule of the present invention is a size sufficient to encode a protein (including a fragment, homologue, or variant of a full-length protein) having biological activity, sufficient to encode a protein comprising at least one epitope which binds to an antibody, or sufficient to form a probe or oligonucleotide primer that is capable of forming a stable hybrid with the complementary sequence of a nucleic acid molecule encoding a natural protein (e.g., under moderate, high, or high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein encoding sequence, a nucleic acid sequence encoding a full-length protein (including a gene), including any length fragment between about 20 nucleotides and the number of nucleotides that make up the full length cDNA encoding a protein, in whole integers (e.g., 20, 21, 22, 23, 24, 25 . . . nucleotides), or multiple genes, or portions thereof.

The phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

In one embodiment, the polynucleotide probes or primers of the invention are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate such as: artificial membranes, organic supports, biopolymer supports and inorganic supports.

One embodiment of the present invention relates to a recombinant nucleic acid molecule which comprises the isolated nucleic acid molecule described above which is operatively linked to at least one expression control sequence. More particularly, according to the present invention, a recombinant nucleic acid molecule typically comprises a recombinant vector and any one or more of the isolated nucleic acid molecules as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remains separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest, such as an enzyme of the present invention). In this embodiment, a nucleic acid sequence encoding the product to be produced (e.g., the protein or homologue or variant thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

Typically, a recombinant nucleic acid molecule includes at least one nucleic acid molecule of the present invention operatively linked to one or more expression control sequences (e.g., transcription control sequences or translation control sequences). As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced. Transcription control sequences may also include any combination of one or more of any of the foregoing.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is generally used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants and describes an inherited change due to the acquisition of exogenous nucleic acids by the microorganism that is essentially synonymous with the term “transfection.” Transfection techniques include, but are not limited to, transformation, particle bombardment, electroporation, microinjection, lipofection, adsorption, infection and protoplast fusion.

One or more recombinant molecules of the present invention can be used to produce an encoded product (e.g., a protein) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., filamentous fungi or yeast or mushrooms), algal, plant, insect, or animal cell that can be transfected. Host cells can be either untransfected cells or cells that are already transfected with at least one other recombinant nucleic acid molecule.

Suitable cells (e.g., a host cell or production organism) may include any microorganism (e.g., a bacterium, a protist, an alga, a fungus, or other microbe), and is preferably a bacterium, a yeast or a filamentous fungus. Suitable bacterial genera include, but are not limited to, Escherichia, Bacillus, Lactobacillus, Pseudomonas and Streptomyces. Suitable bacterial species include, but are not limited to, Escherichia coli, Bacillus subtilis, Bacillus licheniformis, Bacillus stearothermophilus, Lactobacillus brevis, Pseudomonas aeruginosa and Streptomyces lividans. Suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, S chizosaccharomyces pombe, Candida albicans, Hansenula polymorpha, Pichia pastoris, P. canadensis, Kluyveromy ces marxianus and Phaffia rhodozyma.

Suitable fungal genera include, but are not limited to, Chrysosporium, Thielavia, Thermomyces, Thermoascus, Neurospora, Aureobasidium, Filibasidium, Piromyces, Corynascus, Cryptococcus, Acremonium, Tolypocladium, Scytalidium, Schizophyllum, Sporotrichum, Penicillium, Gibberella, Myceliophthora, Mucor, Aspergillus, Fusarium, Humicola, Talaromyces and Trichoderma, and anamorphs and teleomorphs thereof. Suitable fungal species include, but are not limited to, Aspergillus niger, Aspergillus oryzae, Aspergillus nidulans, Aspergillus japonicus, Absidia coerulea, Rhizopus oryzae, Chrysosporium lucknowense, Neurospora crassa, Neurospora intermedia, Trichoderma reesei, Penicillium canescens, Penicillium solitum, Penicillium funiculosum, and Talaromyces flavus. In one embodiment, the host cell is a fungal cell of the species Chrysosporium lucknowense. In another embodiment, a while (low cellulose) strain is sued. In one embodiment, the host cell is a fungal cell of Strain C1 (VKM F-3500-D) or a mutant strain derived therefrom (e.g., UV13-6 (Accession No. VKM F-3632 D); NG7C-19 (Accession No. VKM F-3633 D); UV18-25 (VKM F-3631D), W1L (CBS122189), or W1L# 100L (CBS122190)). Host cells can be either untransfected cells or cells that are already transfected with at least one other recombinant nucleic acid molecule. Additional embodiments of the present invention include any of the genetically modified cells described herein.

In another embodiment, suitable host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly human, simian, canine, rodent, bovine, or sheep cells, e.g. NIH3T3, CHO (Chinese hamster ovary cell), COS, VERO, BHK, HEK, and other rodent or human cells).

In one embodiment, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole, either of which can be used in a composition.

Microorganisms used in the present invention (including recombinant host cells or genetically modified microorganisms) are cultured in an appropriate fermentation medium. An appropriate, or effective, fermentation medium refers to any medium in which a cell of the present invention, including a genetically modified microorganism (described below), when cultured, is capable of expressing enzymes useful in the present invention and/or of catalyzing the production of amino acids or lower molecular weight proteins. Such a medium is typically an aqueous medium comprising assimilable carbon, nitrogen and phosphate sources. Such a medium can also include appropriate salts, minerals, metals and other nutrients. Microorganisms and other cells of the present invention can be cultured in conventional fermentation bioreactors. The microorganisms can be cultured by any fermentation process which includes, but is not limited to, batch, fed-batch, cell recycle, and continuous feimentation. The fermentation of microorganisms such as fungi may be carried out in any appropriate reactor, using methods known to those skilled in the art. For example, the fermentation may be carried out for a period of 1 to 14 days, or more preferably between about 3 and 10 days. The temperature of the medium is typically maintained between about 25 and 50° C., and more preferably between 28 and 40° C. The pH of the fermentation medium is regulated to a pH suitable for growth and protein production of the particular organism. The fermentor can be aerated in order to supply the oxygen necessary for fermentation and to avoid the excessive accumulation of carbon dioxide produced by fermentation. In addition, the aeration helps to control the temperature and the moisture of the culture medium. In general the fungal strains are grown in fermentors, optionally centrifuged or filtered to remove biomass, and optionally concentrated, formulated, and dried to produce an enzyme(s) or a multi-enzyme composition that is a crude fermentation product. Particularly suitable conditions for culturing filamentous fungi are described, for example, in U.S. Pat. No. 6,015,707 and U.S. Pat. No. 6,573,086, supra.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilization.

Proteins of the present invention are preferably retrieved, obtained, and/or used in “substantially pure” form. As used herein, “substantially pure” refers to a purity that allows for the effective use of the protein in any method according to the present invention. For a protein to be useful in any of the methods described herein or in any method utilizing enzymes of the types described herein according to the present invention, it is substantially free of contaminants, other proteins and/or chemicals that might interfere or that would interfere with its use in a method disclosed by the present invention (e.g., that might interfere with enzyme activity), or that at least would be undesirable for inclusion with a protein of the present invention (including homologues and variants) when it is used in a method disclosed by the present invention (described in detail below). Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein of interest is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites), modification of nucleic acid molecules to correspond to the codon usage of the host cell, and deletion of sequences that destabilize transcripts.

Another aspect of the present invention relates to a genetically modified microorganism that has been transfected with one or more nucleic acid molecules of the present invention. As used herein, a genetically modified microorganism can include a genetically modified bacterium, alga, yeast, filamentous fungus, or other microbe. Such a genetically modified microorganism has a genome which is modified (i.e., mutated or changed) from its normal (i.e., wild-type or naturally occurring) form such that the desired result is achieved (i.e., increased or modified activity and/or production of at least one enzyme or a multi-enzyme composition for the degradation of proteins). Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press or Molecular Cloning: A Laboratory Manual, third edition (Sambrook and Russel, 2001), (jointly referred to herein as “Sambrook”). A genetically modified microorganism can include a microorganism in which nucleic acid molecules have been inserted, deleted or modified (i.e., mutated; e.g., by insertion, deletion, substitution, and/or inversion of nucleotides), in such a manner that such modifications provide the desired effect within the microorganism.

In one embodiment, a genetically modified microorganism can endogenously contain and express an enzyme or a multi-enzyme composition for the degradation of protein, and the genetic modification can be a genetic modification of one or more of such endogenous enzymes, whereby the modification has some effect on the ability of the microorganism to degrade protein (e.g., increased expression of the protein by introduction of promoters or other expression control sequences, or modification of the coding region by homologous recombination to increase the activity of the encoded protein).

In another embodiment, a genetically modified microorganism can endogenously contain and express an enzyme for the degradation of protein, and the genetic modification can be an introduction of at least one exogenous nucleic acid sequence (e.g., a recombinant nucleic acid molecule), wherein the exogenous nucleic acid sequence encodes at least one additional enzyme useful for the degradation of protein and/or a protein that improves the efficiency of the enzyme for the degradation of protein. In this aspect of the invention, the microorganism can also have at least one modification to a gene or genes comprising its endogenous enzyme(s) for the conversion of degradation of protein.

In yet another embodiment, the genetically modified microorganism does not necessarily endogenously (naturally) contain an enzyme for the degradation of protein, but is genetically modified to introduce at least one recombinant nucleic acid molecule encoding at least one enzyme or a multiplicity of enzymes for the degradation of protein. Such a microorganism can be used in a method of the invention, or as a production microorganism for crude fermentation products, partially purified recombinant enzymes, and/or purified recombinant enzymes, any of which can then be used in a method of the present invention.

Once the proteins (enzymes) are expressed in a host cell, a cell extract that contains the activity to test can be generated. For example, a lysate from the host cell is produced, and the supernatant containing the activity is harvested and/or the activity can be isolated from the lysate. In the case of cells that secrete enzymes into the culture medium, the culture medium containing them can be harvested, and/or the activity can be purified from the culture medium. The extracts/activities prepared in this way can be tested using assays known in the art. Accordingly, methods to identify multi-enzyme compositions capable of degrading protein are provided.

Antibodies

Another embodiment of the present invention relates to an isolated binding agent capable of selectively binding to a protein of the present invention. Suitable binding agents may be selected from an antibody, an antigen binding fragment, or a binding partner. The binding agent selectively binds to an amino acid sequence selected from Sequences PR 1-PR 430, including to any fragment of any of the above sequences comprising at least one antibody binding epitope.

According to the present invention, the phrase “selectively binds to” refers to the ability of an antibody, antigen binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention. Methods for the generation and production of antibodies are well known in the art.

Monoclonal antibodies may be produced according to the methodology of Kohler and Milstein (Nature 256:495-497, 1975). Non-antibody polypeptides, sometimes referred to as binding partners, are designed to bind specifically to a protein of the invention. Examples of the design of such polypeptides, which possess a prescribed ligand specificity are given in Beste et al. (Proc. Natl. Acad. Sci. 96:1898-1903, 1999). In one embodiment, a binding agent of the invention is immobilized on a substrate such as: artificial membranes, organic supports, biopolymer supports and inorganic supports such as for use in a screening assay.

The present invention is not limited to fungi and also contemplates genetically modified organisms such as algae, bacteria, and plants transformed with one or more nucleic acid molecules of the invention. The plants may be used for production of the enzymes. Methods to generate recombinant plants are known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10:1 (1991). Descriptions of Agrobacterium vector systems and methods for Agrobacterium-mediated gene transfer are provided by numerous references, including Gruber et al., supra, Miki et al., supra, Moloney et al., Plant Cell Reports 8:238 (1989), and U.S. Pat. Nos. 4,940,838 and 5,464,763.

Another generally applicable method of plant transformation is microprojectile-mediated transformation wherein DNA is carried on the surface of microprojectiles. The expression vector is introduced into plant tissues with a biolistic device that accelerates the microprojectiles to speeds sufficient to penetrate plant cell walls and membranes. Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad, Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

Some embodiments of the present invention include genetically modified organisms comprising at least one nucleic acid molecule encoding at least one enzyme of the present invention, in which the activity of the enzyme is downregulated. The downregulation may be achieved, for example, by introduction of inhibitors (chemical or biological) of the enzyme activity, by manipulating the efficiency with which those nucleic acid molecules are transcribed, the efficiency with which the resultant transcripts are translated, and the efficiency of post-translational modifications, or by “knocking out” the endogenous copy of the gene. A “knock out” of a gene refers to a molecular biological technique by which the gene in the organism is made inoperative, so that the expression of the gene is substantially reduced or eliminated. Alternatively, in some embodiments the activity of the enzyme may be upregulated. The present invention also contemplates downregulating activity of one or more enzymes while simultaneously upregulating activity of one or more enzymes to achieve the desired outcome.

The foregoing description of the present invention has been presented for purposes of illustration. The description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### EXAMPLES

### Materials and Methods for Examples 1-6

Enzyme Purification

All purification steps were performed using an ÄKTA explorer P-900 liquid chromatography system (GE Healthcare, Uppsala, Sweden). Separation was done at room temperature and the fractions were collected on ice with an automated fraction collector. Elution was followed at 214 and 280 nm. The protein composition was verified by SDS-PAGE. Abn1 and Abn2 activities were determined on linear arabinan with the PAHBAH assay. Abn4 activity was determined using pNP-arabinofuranoside.

Substrates and Other Materials

Characterization of the C1 arabinohydrolases was performed on linear arabinose oligomers (Megazyme; Bray, Ireland); linear and branched sugar beet arabinan (British Sugar; Peterborough, United Kingdom). Table 1 shows the sugar composition of linear and branched arabinan. To determine side activities, purified fractions were tested on konjac glucomannan (Kalys; Bernin, France), arabinogalactan type II (Meyhall Chemical; Thurgau, Switzerland), Tamarind xyloglucan (Dainippon Pharmaceutical; Osaka, Japan), potato galactan and wheat arabinoxylan (both from Megazyme). Other chemicals were from Sigma-Aldrich or Merck.

TABLE 1
Sugar composition (w/w %) of linear and branched arabinan

| | Rha | Ara | Gal | Glc | GalA | Total Sugar |
|---|---|---|---|---|---|---|
| branched | 3.5 | 65.7 | 14.1 | 4.4 | 9.8 | 97.6 |
| arabinan | | | | | | |
| linear | 4.2 | 55.9 | 18.9 | 6.9 | 13.6 | 99.5 |
| arabinan | | | | | | |

Determination of Protein Concentration

The protein concentrations of the enzyme fractions were determined using the Pierce BCA protein assay kit according to the manufacturer's manual. The protein content was calculated based on a standard curve established with bovine serum albumin (5 to 250 μg/ml). The microtiter plate protocol of the manufacturer was used.

Anion Exchange Chromatography

All enzymes were subjected to Anion Exchange Chromatography (AEC) on a Source 15Q column (50/6; GE Healthcare, self-packed) and eluted at 20 ml/min. The samples were dialyzed against 10 mM sodium phosphate buffer (pH 7.0) overnight at 4° C. and 15 ml sample was loaded onto the column at 5 ml/min. All enzymes were eluted using a sodium chloride (NaCl) gradient in 10 mM sodium phosphate buffer (pH 7.0) comprising 5 segments: 0 mM NaCl for 4 column volumes (CV), a gradient of 0-500 mM NaCl over 20 CV, 500 mM NaCl for 4 CV, 1 M NaCl for 5 CV and 0 mM NaCl for 5 CV (equilibration). Abn2 and Abn4 were eluted with 40 mM to 65 mM NaCl and 43 mM to 129 mM NaCl, respectively. Abn1 did not bind to the column. However, a large amount of protein was bound during AEC and the unbound Abn1 was significantly purified. Also, Abn1 did not bind to cation exchange medium (Source 15S at pH 4.0). Fractions of 20 ml were collected for all samples.

Hydrophobic Interaction Chromatography

Abn1 and Abn2 were further purified by HIC using a HiLoad Phenylsepharose HP 26/10 column (GE Healthcare). The active AEC fractions were pooled and mixed 1:1 with 2.4 M ammonium sulfate in 20 mM Bis-Tris/HCl buffer (pH 6.0) and loaded at 5 ml/min. The samples were eluted using a decreasing ammonium sulfate gradient in 10 mM Bis-Tris/HCl buffer (pH 6.0) comprising 4 segments: 1.2 M ammonium sulfate for 5 CV, a gradient of 1.2-0 M ammonium sulfate over 20 CV, 0 M ammonium sulfate for 5 CV and 1.2 M ammonium sulfate for 5 CV (equilibration). Abn1 and Abn2 were both eluted with 0.9 M to 0.72 M ammonium sulfate. Fractions of 20 ml were collected. The Abn2 containing fractions were pooled and dialyzed at 4° C. overnight against the elution buffer containing 50 mM NaCl (V=5 L) and stored at 4° C.

Size Exclusion Chromatography

After HIC, active Abn1 and Abn4 protein fractions were separately pooled and concentrated using an Amicon ultrafiltration device (Billerica, Mass., USA) with a 12 kDa cutoff membrane. Concentrated samples (5 ml) were subjected to SEC on a preparative Superdex75 column (TK 26/100, GE Healthcare) and eluted at 5 ml/min with 10 mM Bis-Tris/HCl (pH 6.0) containing 50 mM NaCl. Fractions of 5 ml were collected. Purified and active fractions were pooled and stored at 4° C.

SDS-PAGE and Isoelectric Focusing

SDS-PAGE was performed with Biorad mini-protean II system and Biorad Powerpac 300 power supply (Hercules, Calif., USA). Pierce Tris-Hepes SDS gels (12%) were used according to the manufacturer's protocol. Coomassie staining was done over night using the Fermentas PAGE blue stain. Isoelectric focusing with silver staining was performed using the Phast system (GE Healthcare) according to the manufacturer's manual.

Enzyme Incubations

All incubations were carried out at 30° C. unless otherwise mentioned. For biochemical characterization 0.5% (w/v) substrate was used with 0.02% (w/w on protein basis) enzyme. Specific activities were determined towards linear arabinan (Abn1 and Abn2) and branched arabinan (Abn4). Substrates were dissolved in buffer at 60° C. Diluted McIlvaine buffers (20 mM citric acid and 40 mM disodium hydrogen phosphate mixed to give pH 3.0 to pH 8.0) were used to study pH optima and stability. Temperature optima and activity assays were performed in 50 mM sodium acetate buffers (pH 4.5 for Abn2 and pH 5.5 for Abn1 and Abn4) from 20 to 70° C. For end product release linear and branched arabinan (5 mg/ml) were incuated with 0.1 U/ml of the enzymes. Aliquots were taken at 2, 24, 48 and 72 h with 0.1 U/ml additional enzyme at both 24 h and 48 h incubation time. The degradation was followed by high performance size exclusion chromatography (HPSEC). The activity on arabinose monomers and oligomers in the range of DP 2-6 was tested (5 mg/ml; 0.1 U/ml enzyme, t=2, 24 and 48 h with additional 0.1 U/ml enzyme after 24 h), wherein DP=degree of polymerization (e.g., DP2 means arabinobiose). Products were quantified by high performance anion exchange chromatography (HPAEC) with a calibration curve (2-40 pg/ml) of arabinose monomer and oligomers (DP 2-6).

Determination of Reducing Ends with PAHBAH Assay

PAHBAH reducing end assay was performed as described (Lever, 1972). To prepare the working solution one part of p-hydroxybenzoic acid hydrazide (5% w/v) in 0.5 M HCl was mixed with four parts of 0.5 M NaOH. The sample (10 μl) was mixed with 200 μl working solution and incubated at 70° C. for 30 minutes in microtiter plates covered with aluminum foil. After cooling the microtiter plate was centrifuged at 1000 g for 2 min and the absorbance was measured at 405 nm. The reducing end concentration was quantified using an L-arabinose calibration curve (5-750 pg/ml).

Activity Towards p-nitrophenyl-arabinofuranoside

Activity of Abn4 towards p-nitrophenyl-arabinofuranoside (pNP-Ara) was monitored by the release of p-nitrophenol. The sample (10 μl) was incubated with 190 μl pNP-Ara (0.5 mM) in 10 mM sodium acetate buffer (pH 4.5) for one hour at 32° C. The pH was adjusted to pH 7.4 with 50 μl sodium phosphate buffer (0.25 M, pH 7.4). The absorbance was measured at 405 nm. Arabinose release was quantified indirectly with a p-nitrophenol standard curve (10-500 μM).

Sugar Composition Analysis

Polysaccharides were hydrolysed with aqueous 72% (w/w) H₂SO₄ (1 h, 30° C.), followed by hydrolysis with 1 M H₂SO₄ (3 h, 100° C.). Alditol acetates derivatisation was performed as described (Englyst and Cummings, 1984). A Thermo Focus GC gas chromatograph equipped with a Supelco SP 2380 column was used with Helium as inert gas, 24 PSI pressure and a flow rate of 1.1 ml/min. All GC runs were performed using a 2 μl injection volume of sample dissolved in acetone.

Uronic acid content was determined according to Ahmed and Labavitch (1978) using a Skalar Autoanalyzer (Skalar Analytical, Breda, The Netherlands). A galacturonic acid standard curve (12.5-100 μg/ml) was used for quantification.

HPSEC

HPSEC was performed on a Thermo Scientific spectra quest HLPC (Thermo Finnigan, Waltham, Mass., USA) equipped with a set of 4 TSK-Gel G columns (Tosoh bioscience, Tokyo, Japan) in series: guard column PWXL (6 mm ID×40 mm) and separation columns 4000 PWXL, 3000 PWXL and 2500 PWXL (7.8 mm ID×300 mm). Samples (20 μl; 5 mg/me were eluted with filtered aqueous 0.2 M sodium nitrate at 40° C. and a flow rate of 0.8 ml/min. Elution was followed by Refractive index detection (Shodex RI 101; Showa Denko K. K., Kawasaki, Japan).

HPAEC

The monomer and oligomer sugar levels of the digests were analyzed by HPAEC according to Albrecht and co-workers (2009). Arabinose and arabinose oligomers (V=10 μl; c=50-100 μg/ml) were eluted with a different sodium acetate (NaOAc) gradient: 0 mM NaOAc for 5 min, a gradient of 0-500 mM NaOAc over 25 mM, 1 M NaOAc for 10 min and 0 M NaOAc for 15 min (equilibration).

### Example 1

### Purification of Enzymes

Enzymes were purified from crude C1 fermentation liquids of homologous over-expressed enzymes in a C1 empty host strain W1L#100L (Accession No. CBS122190)

Abn1 has a theoretical molecular mass of 32 kDa. It has high sequence similarity with endoarabinanases from glycoside hydrolase (GH) family 43. Abn2, with a theoretical molecular mass of 40 kDa, shows homology with GH family 93 exoarabinanases. Abn4 has a theoretical molecular mass of 33 kDa and high levels of homology with GH43 arabinanases. Abf3 was purified and described to be an arabinoxylan arabinofuranohydrolase by Hinz et al. (2009) using hydrophobic interaction chromatography (HIC, SP Sepharose FF) and size exclusion chromatography (SEC, Superdex 200).

The purification required up to 3 chromatography steps with final recoveries up to 50% in activity. All purified fractions show a single dominant band on SDS-PAGE displaying the protein of interest (data not shown). The molecular masses of the proteins were estimated close to the sequence based values for Abn2 (40 kDa) and Abn4 (33 kDa). For Abn1 a molecular mass of 36 kDa was estimated, which is slightly higher than theoretically expected (32 kDa). This difference may reflect glycosylation of the protein. Glycosylation has been reported for Aspergillus niger endoarabinanase AbnA (Flipphi et al., 1993).

### Example 2

### Biochemical Characterization of Purified Arabinohydrolases

The arabinohydrolases described in the present invention have broad pH optima and stabilities and optimal temperatures of around 50° C. The temperature properties are similar to those reported for other arabinohydrolases. In contrast, the C1 arabinohydrolases act at a higher pH and in a broader range than most fungal arabinohydrolases. Interestingly, their pH optima are similar to those of most bacterial arabinohydrolases (Beldman et al., 1997; Saha, 2000). Considering the agreement between the pH optima of the arabinohydrolases and the pH optimum of typical yeasts, these data reveal that the C1 arabinohydrolases can be highly useful in the liquefaction of sugar beet pulp for bioethanol production.

pH and Temperature Optima

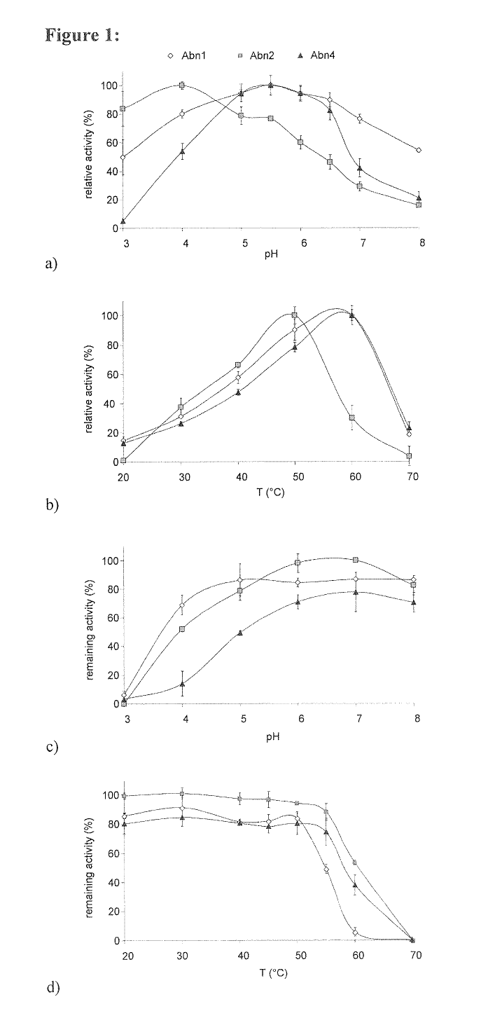

The pH optima determined for Abn1, Abn2 and Abn4 are illustrated in FIG. 1A. All enzymes are most active under slightly acidic conditions. Abn1 and Abn4 are most active between pH 5.0 and 6.5 with a maximum at pH 5.5. The Abn2 activity is highest between pH 3.0 and 5.5 with a maximum at pH 4.0. All enzymes have relatively broad optima. Hence, they can potentially degrade arabinan jointly in a single incubation.

In FIG. 1B the temperature optima of Abn1, Abn2 and Abn4 are shown. The temperature optimum is 50° C. for Abn2 and 60° C. for Abn1 and Abn4. The optimum curves for all enzymes are asymmetric with a nearly twofold increase per 10K temperature increment from 20° C. to 50° C. Above optimum temperatures the enzyme activities rapidly decrease. For arabinoxylan arabinofuranohydrolase Abf3 optimal reaction rates have been reported at 40° C. and pH 5.0. The enzyme was stable up to 50° C. and completely inactivated above 65° C. (Hinz et al. 2009, Pouvreau et al. 2009).

pH and Temperature Stabilities

FIG. 1C shows the pH stability of Abn1, Abn2 and Abn4. It can be seen that the curves of all enzymes are relatively broad. All enzymes are unstable at pH 3.0 or lower and show different stabilities between pH 4.0 and 8.0. Abn1 is very stable between pH 5.0 and pH 8.0 and even possesses 70% of its optimal activity at pH 4.0. Abn2 has similar pH stability as Abn1, but the stability has a more pronounced optimum at pH 6.0 to 7.0. Abn4 is stable in the neutral pH range between pH 6.0 and 8.0, however, the remaining activity is only 80% indicating that Abn4 is less stable than Abn1 and Abn2.

The temperature stabilities of Abn1, Abn2 and Abn4 are presented in FIG. 1D. All three enzymes are stable up to 50° C. with Abn2 and Abn4 showing a slightly higher stability up to 55° C. The remaining activity of Abn1 is 85% of the optimal activity up to 50° C. and is almost lost 60° C. Abn2 is the most stable enzyme having 90% of its initial activity at 55° C. It is completely inactivated at 70° C. and above. Abn4 behaves similarly with the difference that, even at 20° C., only 80% of the initial activity could be recovered. Long term stability for all enzymes was tested over 24 hours at pH 6.0 and 30° C. It was found that Abn1 and Abn2 enzymes remain active to more than 90% and Abn4 still had 80% of its initial activity (no further data shown).

Specific Activities

The specific activities of purified Abn1 and Abn2 towards linear arabinan are 26 U/mg and 7.1 U/mg, respectively. Abn4 has a specific activity of 9.5 U/mg towards branched arabinan. These activities are in the same order of magnitude as reported for many arabinohydrolases from other sources (de Vries et al., 2000; Skjot et al., 2001). Purified Abn1, Abn2 and Abn4 did not show activity against oat spelt xylan, wheat arabinoxylan, arabinogalactan type II, potato galactan, konjac glucomannan, polygalacturonic acid, carboxymethyl cellulose and tamarind xyloglucan.

### Example 3

### Enzyme Specificity Towards Natural Substrates: Actions on Arabinose Oligomers

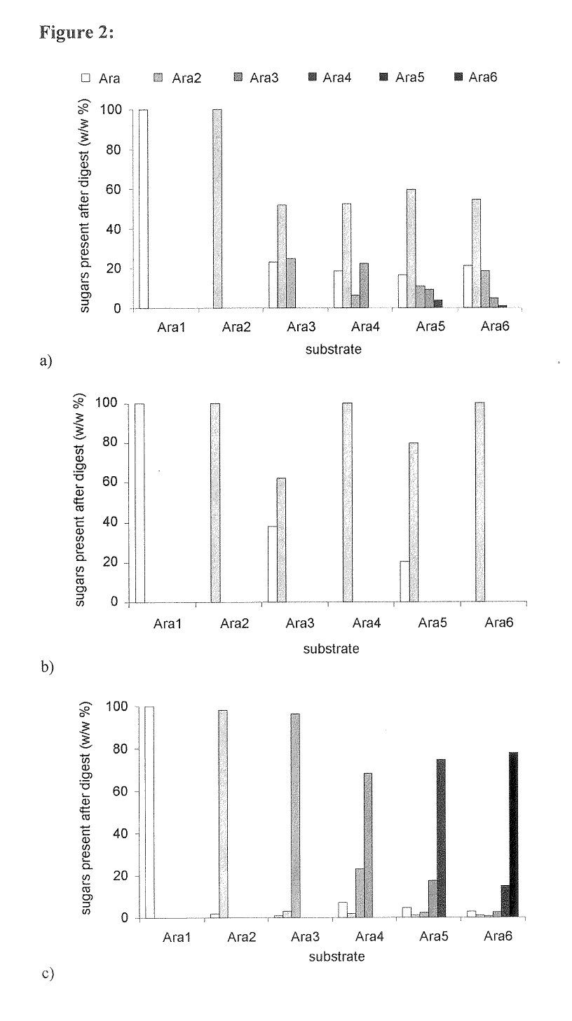

The performance of the C1 arabinohydrolases was tested on linear arabinose oligomers ranging from DP 2-6. FIG. 2A shows that Abn1 degrades oligomers in the range from DP 3-6 and produces, on a weight basis, 50-60% arabinobiose and 20% arabinose monomers. At the end point of the digestion 25% of the oligomers remain present with DP≧3. Arabinotriose was the main product from arabinohexaose after 2 h (data not shown). This indicates an unspecific exo mode of action or an endo mode of action with preference for larger oligomers, as also described for Aspergillus niger endoarabinanase (Rombouts et al., 1988).

Abn2 is active on linear arabinose oligomers starting from arabinotriose (FIG. 2b). It splits off an arabinobiose unit from the trimer. Arabinotetraose and arabinohexaose are fully converted into arabinobiose. From arabinotriose and arabinopentaose arabinose monomers are left over after releasing dimer from the oligomer. No other oligomers are released at any stage of digestion indicating that Abn2 is an arabinobiose releasing exoarabinanase.

Abn4 is not as active towards arabinobiose and arabinotriose, leaving more than 90% of the substrate unaltered (FIG. 2C). In contrast, Abn4 could remove arabinose monomers from DP 4-6 oligomers. However, this activity is rather low, leaving more than 60% of the substrates undigested.

The arabinoxylan arabinofuranohydrolase Abf3 was also tested on arabinose oligomers. It is very active and completely hydrolyzed all oligomers into arabinose monomers (data not shown; See Kühnel et al 2011. Bioresource Technology 102; 1636-1643).

### Example 4

### Enzyme Specificity Towards Natural Substrates: Molecular Mass Distribution Upon Maximal Product Conversion

Linear Arabinan

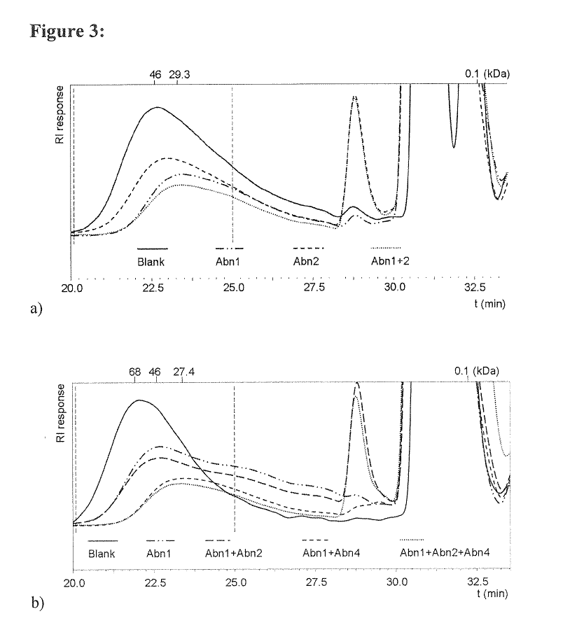

The molecular mass distributions of linear and branched arabinan after different enzyme digestions are presented in FIG. 3. When digested with Abn1, the average molecular mass of the high molecular mass fraction between 20 and 25 min (HMM) shifts from 46 to 30 kDa with a concomitant decrease of the peak area by 60% (FIG. 3a). The 30 kDa peak remains in both, linear and branched arabinan digestions. It could reflect a rhamnogalacturonan I core structure, to which the arabinan side chains are bound to. It can be seen from Table 1 that linear and branched arabinans contain considerable amounts of rhamnose, galacturonic acid and galactose (32 and 37% (w/w), respectively) that are likely to be part of RG I. Therefore, the 60% decrease in the HMM peak area suggests that Abn1 can efficiently cut the backbone of linear arabinan and degrade the polymers to small molecular mass oligomers. Abn2 decreases the peak area of the HMM fraction by 40%, while it maintains its average molecular mass. This result is confirming the exo mode of action of Abn2. A combined digestion with Abn1 and Abn2 results in the strongest degradation and a 67% HMM peak area decrease is observed. Abn2 digests contain an additional peak at 29 min derived from ammonium sulfate, which was not fully removed after hydrophobic interaction chromatography.

Branched Arabinan

When branched arabinan is incubated with Abn1, the average molecular mass of the HMM fraction shifts from 68 to 46 kDa, while its area decreases by 30% (FIG. 3b). The broadened mass distribution indicates that Abn1 cuts the substrate only one or two times, suggesting that Abn1 is hindered by arabinose side chains. A combined digestion with Abn1 and Abn2 results in a similar pattern. This combination can degrade 10% more polymeric arabinan than Abn1 alone.

Abn4 is active on branched arabinan. However, it only slightly influences the average molecular mass distribution and peak area (not shown). A combination of Abn1 and Abn4 degrades 65% of the branched arabinan. A combination of all 3 enzymes degrades 70% of the arabinan polymer and, as seen for linear arabinan, decreases the remaining average molecular mass to approximately 30 kDa.

It can be concluded that effective degradation of linear arabinan requires Abn1, whereas a combination of Abn1 and Abn4 is needed for the degradation of branched arabinan. Abn2 slightly enhances the degradation of both substrates.

### Example 5

### Enzyme Specificity Towards Natural Substrates: End Product Release

Linear Arabinan

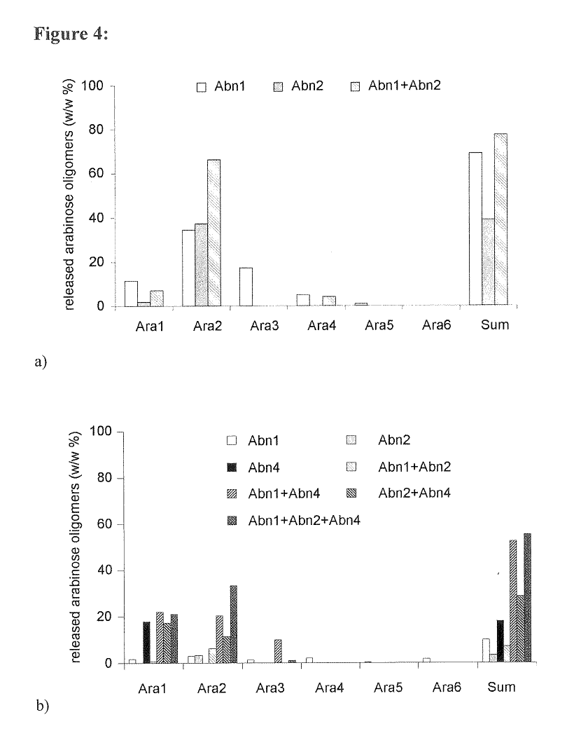

The hydrolysis products after maximal substrate conversion were analyzed and quantified. The oligomer release from linear arabinan is shown in FIG. 4a. Abn1 releases 69% of the total arabinose as DP 1-4 oligomers, mainly as arabinobiose. Abn2 degrades 40% of the arabinose present in the polymer to arabinobiose. A combination of Abn1 and Abn2 releases almost 80% of the arabinose present. Abn4 does not act on the linear arabinan polymer, neither alone nor combined with Abn1 and Abn2.

The degradation of linear arabinan by Abn1 was also monitored at different times (data not shown). In early stages oligomers in the range of DP 3-15 are produced. These oligomers are mainly broken down to arabinotriose after 24 h and, after 72 h, to arabinobiose and arabinose. A similar pattern was reported for Arabinanase A from Pseudomonas fluorescens (McKie et al., 1997).

The results confirm that Abn1 is an endoarabinanase. Time-dependent degradation data suggest that Abn1 follows a multiple chain attack mechanism with preference for larger oligomers. Unlike Abn1, Abn2 does not produce any oligomers, but only arabinobiose at any stage of the reaction. It is, therefore, confirmed that Abn2 is an exoarabinanase able the release arabinobiose from the α-1,5-arabinan backbone.

Branched Arabinan

The oligomer release from branched arabinan was also quantified upon maximal substrate conversion (FIG. 4b). Abn1 and Abn2 only released on a weight basis 10 and 3% of the total arabinose as linear oligomers, respectively. Both enzymes are hindered by the presence of arabinose side chains. Abf3 alone did not act on branched arabinan. A combination of Abn1 and Abf3 released, on a weight basis, 25% of the arabinose present as monomers (no further data shown). This suggests that Abf3 is not active on arabinan polymers, but it can only act on arabinose oligomers. Abn4 could release 18% of total arabinose as monomers. A combined incubation with Abn1 and Abn4 releases 52% of total arabinose present as arabinose monomers and linear arabinose oligosaccharides. This indicates that Abn4 is an arabinofuranosidase active on the side chains of sugar beet arabinan. The relatively low yield of polymeric arabinan as arabinose monomer and linear oligomers suggests that Abn4, like Aspergillus niger Abf B (Rombouts et al., 1988), cannot hydrolyze all types of linkages present in branched arabinan. More in depth structural analysis is necessary to determine the linkage specificity of Abn4.

### Example 6

### Enzyme Specificity Towards Natural Substrates: Release of Non Linear Arabinose Oligomers

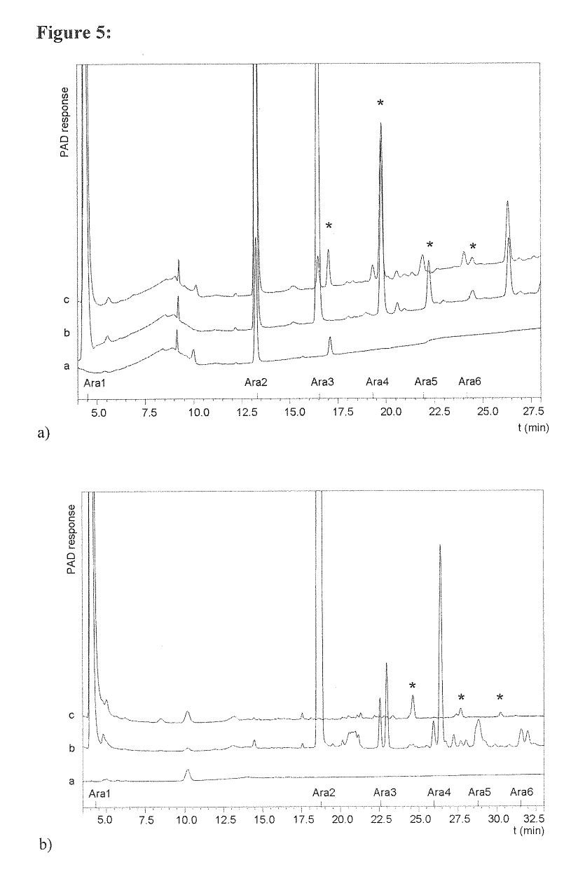

The digest of branched arabinan with Abn1, Abn2 and Abn4 released 56% of the arabinose present as arabinose monomers and linear oligomers. The relatively low oligomer release could be explained by the formation of arabinose isomers as indicated by the HPAEC elution profile of branched arabinan samples treated with C1 arabinohydrolases shown in FIG. 5a. It can be seen that Abn2 alone releases small amounts of arabinobiose and two unknown peaks eluting at 10 and 17 min (line a). Abn1 and Abn4 release high amounts of arabinose, arabinobiose and arabinobiose (line b). Besides linear oligomers a number of unknown peaks (marked by asterisks) appear that elute shortly after the linear standard oligomers (marked by asterisks). A combination of the Abn1, Abn2 and Abn4 (Abn124) produces a more complex mixture of oligomers (line c). It is likely that these peaks represent isomers of arabinose oligomers. To test this hypothesis arabinofuranosidase Abf3 was added to an Abn124 digest. The samples were analysed at higher concentrations (500-1000 μg/ml) with a less steep gradient than normal to achieve higher sensitivity and better separation (0-350 mM NaOAc in 25 min). From FIG. 5b it can be seen that even more unidentified peaks can be recognized in the Abn124 digest (line b). When Abf3 is added, the majority of the peaks representing both unknown and linear arabinose oligomers are degraded to monomers (line c). This indicates that the unknown peaks are arabinose oligosaccharides as well. It also strengthens the hypothesis that Abn4 does not act on all types of side chain linkages. Adding Abf3 also results in a series of unknown peaks (asterisks), probably derived from higher molecular weight material. This is the first report is the first one that describes the release of isomeric arabinose oligomers by an exoarabinanase.

### Conclusion of Examples 1-6

The arabinohydrolases Abn1, Abn2, Abn4 and Abf3 from Chrysosporium lucknowense act together on the degradation of arabinans. Their activities towards various substrates are summarized in Table 2. It clearly shows the preference the individual enzymes for certain arabinan substructures, such as the degradation of linear regions, branches or oligomers. All enzymes are stable in a wide pH range and resist temperatures up to 50° C., which makes them suitable for arabinan degradation from sugar beet pulp (see Example 2). Endoarabinanase Abn1 and arabinofuranosidase Abn4 release 52% of the arabinose as monomers and linear arabinose oligomers and small amounts of unknown arabinose oligomers (see Examples 4-6). The inclusion of Abn2 results in a release of 56% linear arabinose oligomers and an even broader variety of unknown arabinose oligomers (see Examples 4-6). A yield of 80% is reached, when linear arabinan is degraded with a combination of Abn1 and 2. Abf3 converts all oligomers formed by Abn1, 2 and 4 to arabinose monomers(see Examples 4-6).

TABLE 2

| | Activity of C1 arabinohydrolases towards various substrates. | | | |
|---|---|---|---|---|
| | Linear | Branched | Linear arabinose | p-NP- |
| | arabinan | Arabinan | oligomers | Arabinofuranoside |
| Abn1 | ++ | +/− | + | − |
| Abn2 | + | +/− | + | − |
| Abn4 | − | + | +/− | ++ |
| Abf3 | − | − | ++ | ++ |

### Materials and Methods for Examples 7-8

Materials

Branched sugar beet arabinan was obtained from British Sugar (patent McCleary¹⁷). The arabinose content is 67% (w/w %), the remaining part consists of hairy regions (rha, galA and gal) and glucans (glc)⁸.

Linear arabino-oligosaccharides (DP2-8) have been purchased from Megazyme International Ltd (Bray, Ireland).

Enzymatic Degradation of Sugar Beet Arabinan

For fractionation and isolation of branched AOS two times 1 g of branched sugar beet arabinan have been digested with the arabino-hydrolases Abn1, Abn2 and Abn4 derived from Chrysosporium lucknowense strain C1⁸. One arabinan batch has been incubated with an overdose of Abn4 (1.10 U, t=15 h), whereas another batch has been incubated with 0.22 U Abn4 (t=15 h) resulting in about 30% of maximal Abn4 degradation. The enzyme dosage has been calculated based on the fact that about 18% of arabinose present can be degraded by Abn4⁸. Both incubations wereiollowed by an end-point degradation of Abn1 and Abn2. All enzyme incubations have been performed at 30° C. (pH 5).

High Performance Anion Exchange Chromatography (HPAEC, pH 12)

Arabinose and AOS were determined by HPAEC with pulsed amperometric detection (PAD). A HPAEC system (ICS-3000, Dionex Corporation, Sunnyvale, Calif., USA)) was equipped with a CarboPac PA-1 separation column and a Carbopac PA-1 guard column (2 mm ID×250 mm and 2 mm ID×25 mm; Dionex Corporation). A flow of 0.3 mL/min was used and the temperature was kept at 20° C. AOS (injection volume 10 μL; 10 to 100 μg/mL) were separated using a gradient with 0.1 M NaOH (solution A) and 1 M NaOAc in 0.1 M NaOH (solution B): 0-36 min from 0% B to 42% B, 36-42 min at 100% B and 42-57 min at 0% B.

Fractionation Based on Size: Biogel P2

Fractionation was performed on a Äkta Explorer system (Amersham Biosciences, Uppsala, Sweden) equipped with a Bio-Gel P2 column (porous polyacrylamide, 1000×26 mm, 200-400 mesh, Bio-Rad Laboratories, Hercules, Calif.) thermostated at 60° C. and eluted with Millipore water at 1.0 mL/min. For each sample, 20 mL with a concentration of 50 mg/mL was injected. The column efflux was first led through a refractive index detector (Shodex R172, Showa Denko K. K., Tokyo, Japan) it was collected in fractions of 3.5 mL by a fraction collector (Superfrac, GE Amersham, Uppsala, Sweden). Appropriate fractions were pooled and freeze-dried for further analysis.

Determination of Neutral Sugar and Uronic Acid Content of the Biogel P2 Fractions

The total neutral sugar and uronic acid content were determined with automated colorimetric assay analyzer. The total neutral sugar content has been determined by using the orcinol-sulfuric acid color assay and arabinose (25-200 μg/mL) as standard curve¹⁸. The uronic acid content was determined with the metahydroxy-biphenyl assay and calculated based on a standard curve from 12.5 to 100.0 μg/mL established with galacturonic acid¹⁹.

MALDI-TOF MS

Each sample was desalted with AG 50W-X8 Resin (Bio-Rad Laboratories, Hercules, USA) 1 μL of the desalted sample solution was mixed on a MALDI-plate (Bruker Daltonics, Bremen, Germany) with 1 μL matrix solution of 12 mg/mL 2,5-dihydroxy benzoic acid (Bruker Daltonics) in 30% acetonitrile and dried under a stream of air²⁰. MALDI-TOF MS analysis was performed using an Ultraflex workstation (Bruker Daltonics) equipped with a nitrogen laser of 337 nm and operated in positive mode. After a delayed extraction time of 350 ns, the ions were accelerated to a kinetic energy of 22000 V. The ions were detected using reflector mode. The lowest laser power required to obtain good spectra was used and spectra were collected with each measurement. The mass spectrometer was calibrated with a mixture of maltodextrins (Avebe, Foxhol, The Netherlands; MD20; mass range 500-2000 m/z). The data was processed using Bruker Daltonics flexAnalysis version 2.2.

NMR Analysis

Samples (1-6 mg) have been exchanged with D₂O (99.9 atom %, Sigma-Aldrich, St. Louis, Mo., USA) and subsequently dissolved in 0.5 mL D₂O (99.9 atom %, Sigma Aldrich) containing 0.75% 3-(trimethylsilyl)-propionic-2,2,3,3-d₄ acid, sodium salt (TMSP). NMR spectra were recorded at a probe temperature of 300K on a Bruker Avance-III-600 spectrometer, equipped with a cryo-probe located at Biqualys (Wageningen, The Netherlands). Chemical shifts are expressed in ppm relative to internal TMSP at 0.00 ppm. 1D and 2D COSY, TOCSY, HMBC, and HMQC spectra were acquired using standard pulse sequences delivered by Bruker. For the ¹H-COSY and -TOCSY spectra, 400 experiments of 2 scans were recorded, resulting in measuring times of 0.5 h. The mixing time for the TOCSY spectra was 100 ms. For the [¹H,¹³C]-HMBC and -HMQC spectra 800 experiments of 32 scans and 512 experiments of 8 scans, respectively, were recorded, resulting in measuring times of 8.7 h and 2.5 h, respectively.

### Example 7

### Enzymatic Preparation of Arabino-Oligosaccharides (AOS) from Sugar Beet Arabinan

Enzymatic degradation of sugar beet arabinan with a mixture of the arabino-hydrolases Abn1, Abn2 and Abn4 (Chryosporium lucknowense strain C1) releases the main degradation products arabinose and arabinobiose, but also produces various unknown AOS, which elute differently in high performance anion exchange chromatography with pulsed amperometric detection (HPAEC-PAD) compared to linear α-(1,5)-linked AOS. To explore the precise structure of various unknown AOS, sugar beet arabinan has been digested with two different mixtures of Abn1, Abn2 and Abn4.

Although sugar beet arabinan only contains 66% arabinose (w/w %) in addition to significant amounts of residual rhamnogalacturonan I, the use of pure and well defined arabino-hydrolases ensured specific degradation of the arabinan segments for this experiment. To the first digest (D-30) the arabino-furanosidase Abn4 has been added in a concentration that should ensure partial degradation of the side chains of sugar beet arabinan resulting in partly debranched backbone. From HPAEC results showed that 30% of the maximal degradation by Abn4 took place, taking the arabinose released as a measure for the Abn4 action (data not shown). To the second digest (D-100) Abn4 has been added in an overdose, thus allowing Abn4 to cleave all possible linkages by releasing about 18% of the arabinose present. This results in a heavily debranched arabinan backbone. Both digests were treated subsequently with a mixture of the endo-arabinanase Abn1 and the exo-arabinanase Abn2 to ensure degradation of the linear part of the arabinan present towards mono-, di- and oligosaccharides.

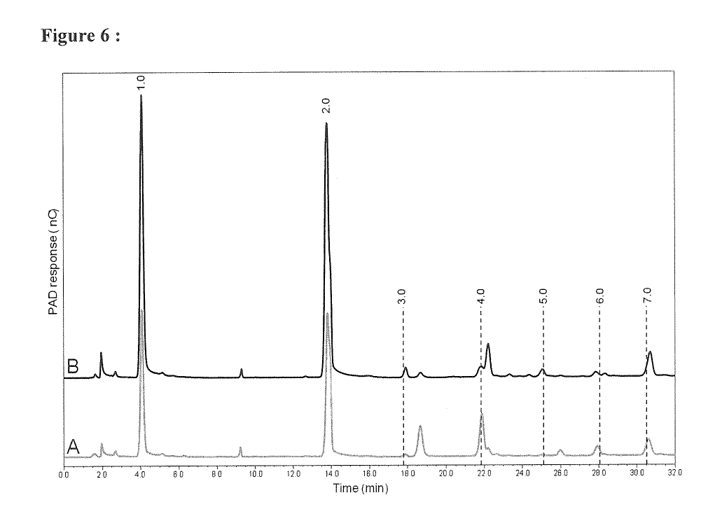

The HPAEC chromatograms of both enzyme digests, D-30 and D-100, are presented in FIG. 6A and FIG. 6B, respectively. In both digests, the main degradation products were arabinose and arabinobiose, which levels increased with an increase of Abn4 conversion level (D-30 to D-100). In addition to the monomer and dimer, several oligomeric structures can be seen as well. Most of these oligomeric structures do not co-elute with the α-(1,5)-linked AOS standards as indicated in FIG. 6, concluding that these peaks are branched AOS as hypothesized already earlier by Kühnel et al. The peak at 18.4 min, which is present in the D-30 digest, and the peak at 22.1 min, which appears in the D-100 digest, are most abundant, although many more unknown AOS are present in minor quantities in both digests. As HPAEC analysis of both digests indicated the presence of various unknown AOS, both digests were subjected to further analysis.

### Example 8

### Fractionation of the Arabino-Oligosaccharides (AOS) of Sugar Beet Arabinan After Enzyme Degradation

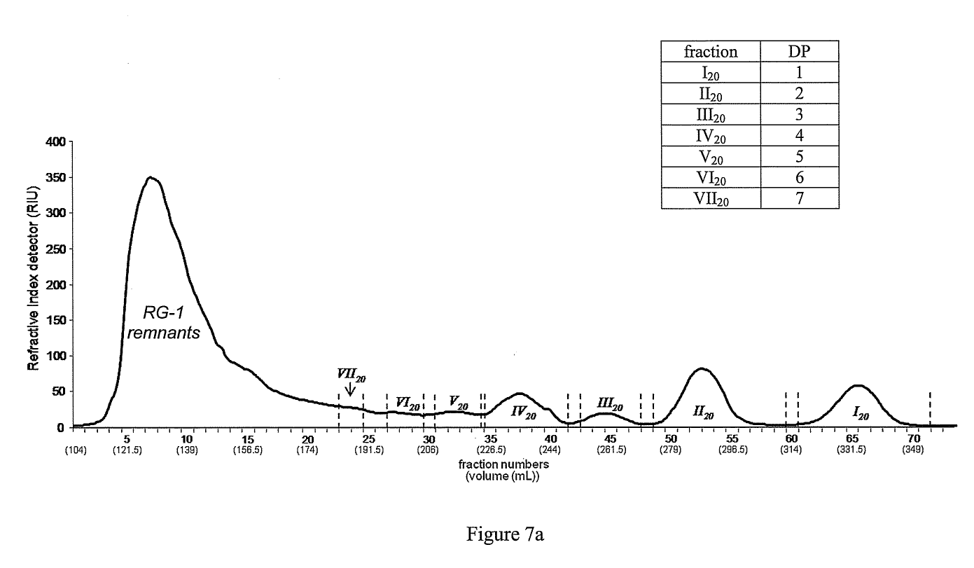

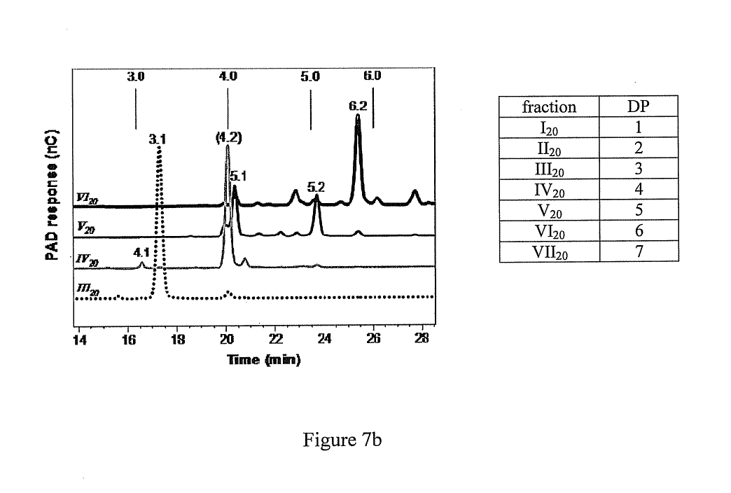

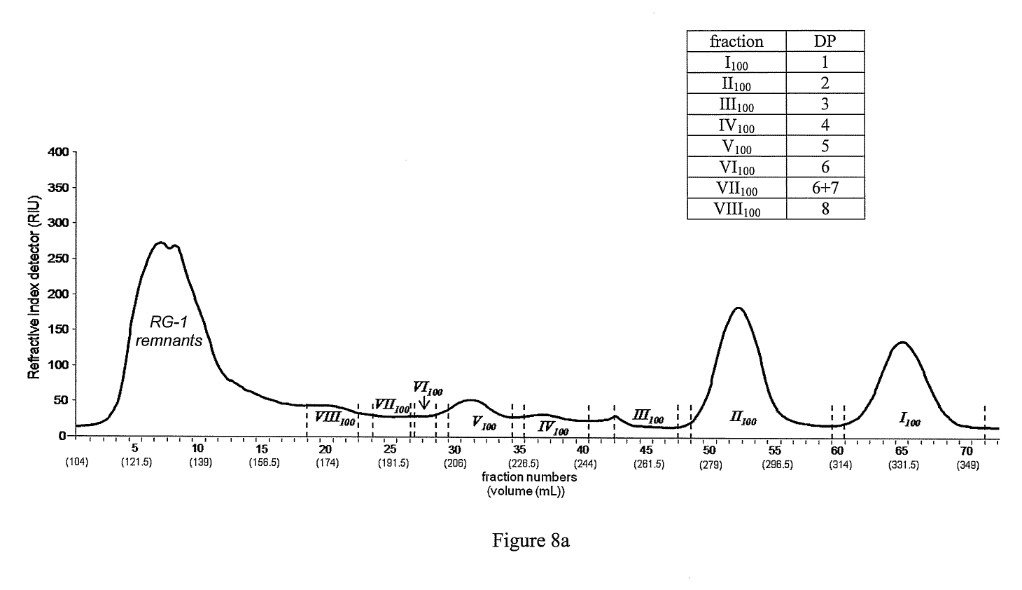

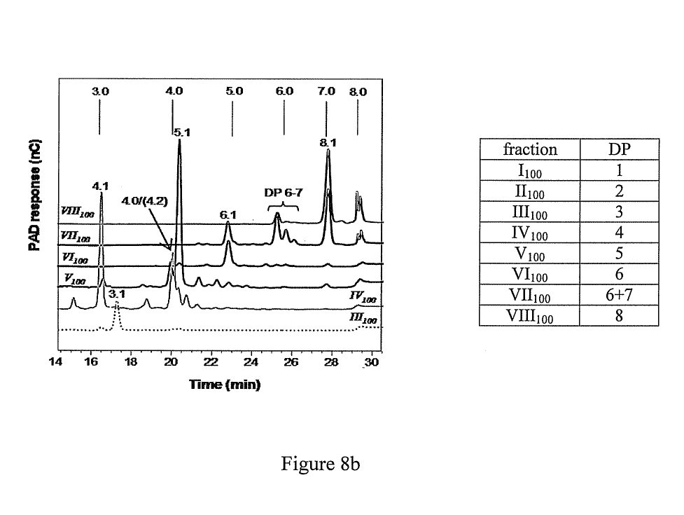

For detailed structural characterization of the AOS, a preparative fractionation based on size of both digests was performed. In FIG. 7A and FIG. 8A the refractive index (RI) patterns of both Biogel P2 separations are given including the DP as established using MALDI-TOF MS analysis (FIG. 7, inserted table). Analysis of the total sugar content of all fractions taken (3.5 mL each) confirmed the RI patterns of both digests. Significant amounts of uronic acid were only detected in the first 15 fractions of the Biogel P2 separations, supporting the assumption that the main peak in both digests in the beginning of the RI patterns is assigned to the remaining rhamnogalacturonan-I (RG-I) core structure (‘RG-I remnants’ in FIG. 7A and FIG. 8A). Neutral fractions (number 18-71) from the Biogel P2 separations (3.5 mL each) have been analyzed by using HPAEC and MALDI-TOF MS. The fractions have been pooled based on HPAEC analysis aiming at pools with high purity. The pool numbers are indicated as I₂₀-VII₂₀ and I₁₀₀-VIII₁₀₀ in FIG. 7A and FIG. 8A for sample D-30 and D-100, respectively.

In the following part HPAEC, MALDI-TOF MS and NMR results of the various pools will be discussed in more detail. Concerning the NMR results of all resolved structures it can be stated that full assignment of both proton and carbon spectra was possible combining the data of the various 2D experiments (Table 3). All linkages could be confirmed with HMBC cross peaks.

TABLE 3
¹H and ¹³C-NMR data of arabino-oligosaccharides identified from sugar beet arabinan;

| | H-1 | H-2 | H-3 | H-4 | H-5R | H-5S | C-1 | C-2 | C-3 | C-4 | C-5 |
|---|---|---|---|---|---|---|---|---|---|---|---|
| Pools II₃₀ | | | | | | | | | | | |
| and II₁₀₀ | | | | | | | | | | | |
| R α | 5.265 | 4.04 | 4.04 | 4.24 | 3.769 | 3.87 | 104.01 | 84.18 | 78.71 | 84.23 | 69.69 |
| R β | 5.306 | 4.10 | 4.10 | 3.95 | 3.769 | 3.86 | 98.15 | 78.63 | 77.19 | 82.21 | 71.03 |
| T | 5.085 | 4.132 | 3.956 | 4.10 | 3.72 | 3.834 | 110.26 | 83.7* | 79.36 | 86.8* | 64.04 |
| Pool III₃₀ | | | | | | | | | | | |
| R α | 5.257 | 4.04 | 4.06 | 4.236 | 3.77 | 3.86 | 103.98 | 84.22 | 78.55 | 84.10 | 69.13 |
| R β | 5.312 | 4.10 | 4.10 | 3.95 | 3.78 | 3.85 | 98.18 | 78.90 | 77.16 | 82.21 | 70.69 |
| A | 5.116 | 4.293 | 4.04 | 4.197 | 3.768 | 3.877 | 110.26 | 82.03 | 84.94 | 86.0* | 63.9 |
| T | 5.17 | 4.144 | 3.948 | 4.04 | 3.717 | 3.842 | 109.89 | 84.05 | 79.38 | 86.73 | 63.99 |
| Pool IV₃₀ | | | | | | | | | | | |
| R α | 5.256 | 4.04 | 4.06 | 4.241 | 3.77 | 3.86 | 103.99 | 84.2 | 78.55 | 84.10 | 69.21 |
| R β | 5.308 | 4.10 | 4.10 | 3.95 | 3.78 | 3.85 | 98.19 | 78.88 | 77.15 | 82.16 | 70.73 |
| A | 5.122 | 4.294 | 4.10 | 4.319 | 3.856 | 3.95 | 110.29 | 81.92 | 85.2 | 84.52 | 69.29 |
| T3 | 5.165 | 4.14 | 3.96 | 4.05 | 3.713 | 3.839 | 109.97 | 84.05 | 79.39 | 86.77 | 63.99 |
| T5 | 5.097 | 4.14 | 3.96 | 4.115 | 3.734 | 3.839 | 110.18 | 83.78 | 79.39 | 86.83 | 63.99 |
| Pool IV₁₀₀ | | | | | | | | | | | |
| R α | 5.26 | 4.04 | 4.06 | 4.242 | 3.78 | 3.86 | 103.99 | 84.17 | 78.58 | 84.10 | 69.21 |
| R β | 5.307 | 4.10 | 4.10 | 3.95 | 3.78 | 3.85 | 98.19 | 78.86 | 77.22 | 82.18 | 70.73 |
| A | 5.253 | 4.31 | 4.19 | 4.19 | 3.77 | 3.89 | 109.14 | 88.0* | 82.9 | 85.4* | 63.54 |
| T2 | 5.189 | 4.13 | 3.97 | 4.08 | 3.727 | 3.84 | 109.86 | 84.1 | 79.31 | 86.98 | 63.93 |
| T3 | 5.165 | 4.15 | 3.96 | 4.05 | 3.718 | 3.84 | 109.7 | 84.02 | 79.37 | 86.83 | 63.97 |
| Pool V₂₀ | | | | | | | | | | | |
| R α | 5.26 | 4.04 | 4.05 | 4.24 | 3.77 | 3.86 | 103.99 | 84.21 | 78.56 | 84.10 | 69.22 |
| R β | 5.3 | 4.10 | 4.10 | 3.96 | 3.78 | 3.85 | 98.17 | 78.89 | 77.20 | 82.20 | 70.75 |
| A | 5.12* | 4.29* | 4.11 | 4.32 | 3.85 | 3.95 | 110.2 | 82.0* | 85.18 | 84.5* | 68.71 |
| T3 | 5.16 | 4.14 | 3.96 | 4.05 | 3.725 | 3.84 | 110.04 | 84.07 | 79.40 | 86.77* | 63.97 |
| B | 5.125 | 4.3 | 4.04 | 4.221 | 3.77 | 3.88 | 110.2 | 82.04 | 84.95 | 86.08 | 63.89 |
| T3 | 5.164 | 4.14 | 3.96 | 4.05 | 3.725 | 3.84 | 109.86 | 84.07 | 79.4 | 86.85* | 63.97 |
| Pool V₁₀₀ | | | | | | | | | | | |
| R α | 5.26 | 4.04 | 4.05 | 4.24 | 3.782 | 3.86 | 104.03 | 84.2 | 78.62 | 84.12 | 69.41 |
| R β | 5.30 | 4.10 | 4.10 | 3.96 | 3.782 | 3.85 | 98.2 | 78.9 | 77.20 | 82.14 | 70.96 |
| A | 5.260 | 4.315 | 4.256 | 4.32 | 3.860 | 3.95 | 109.2 | 87.8* | 83.12 | 84.0 | 68.88 |
| T2 | 5.189 | 4.14 | 3.97 | 4.08 | 3.725 | 3.84 | 109.8 | 84.2 | 79.40 | 86.92 | 63.96 |
| T3 | 5.165 | 4.15 | 3.96 | 4.05 | 3.725 | 3.84 | 109.8 | 84.0 | 79.40 | 86.9 | 63.96 |
| T5 | 5.092 | 4.14 | 3.96 | 4.11 | 3.725 | 3.84 | 110.19 | 83.93 | 79.44 | 86.69 | 63.96 |
| Pool VI₃₀ | | | | | | | | | | | |
| R α | 5.26 | 4.04 | 4.06 | 4.24 | 3.77 | 3.85 | 103.98 | 84.21 | 78.56 | 84.1 | 69.22 |
| R β | 5.31 | 4.10 | 4.10 | 3.95 | 3.77 | 3.85 | 98.20 | 78.88 | 77.20 | 82.21 | 70.80 |
| A | 5.12* | 4.290 | 4.12 | 4.32 | 3.85 | 3.96 | 110.3* | 82.09 | 85.13 | 84.66 | 68.72 |
| T3 | 5.162 | 4.15 | 3.96 | 4.05 | 3.72 | 3.84 | 109.94 | 84.09 | 79.40 | 86.74 | 63.95^{b} |
| B | 5.133 | 4.303 | 4.10 | 4.346 | 3.77 | 3.85 | 110.21 | 81.9 | 85.21 | 84.66 | 69.27 |
| T3 | 5.162 | 4.15 | 3.96 | 4.05 | 3.72 | 3.84 | 109.94 | 84.09 | 79.40 | 86.74 | 63.95^{b} |
| T5 | 5.098 | 4.14 | 3.96 | 4.12 | 3.73 | 3.84 | 110.23 | 83.8 | 79.44 | 86.83 | 64.00^{b} |
| Pool VII₃₀ | | | | | | | | | | | |
| R α | 5.26 | 4.04 | 4.06 | 4.24 | 3.773 | 3.86 | 104 | 84.22 | 78.55 | 84.10 | 69.2 |
| R β | 5.31 | 4.1 | 4.1 | 3.96 | 3.773 | 3.86 | 98.2 | 78.87 | 77.20 | 82.10 | 70.69 |
| A | 5.12* | 4.3 | 4.1 | 4.32 | 3.85 | 3.96 | 110.3^{c} | 81.94^{d} | 85.17 | 84.51 | 69.27^{g} |
| T3 | 5.164 | 4.15 | 3.96 | 4.05 | 3.73 | 3.84 | 109.96 | 84.08 | 79.40 | 86.74^{f} | 63.9^{h} |
| B | 5.09 | 4.14 | 4.05 | 4.23 | 3.807 | 3.901 | 110.3^{c} | 83.72^{e} | 79.37 | 85.08 | 69.07 |
| C | 5.127 | 4.3 | 4.1 | 4.32 | 3.85 | 3.96 | 110.27^{c} | 81.97^{d} | 85.17 | 84.51 | 69.38^{g} |
| T3 | 5.164 | 4.15 | 3.96 | 4.05 | 3.73 | 3.84 | 109.96 | 84.08 | 79.40 | 86.771 | 63.96″ |
| T5 | 5.097 | 4.14 | 3.96 | 4.12 | 3.73 | 3.84 | 110.2 | 83.79^{e} | 79.44 | 86.83 | 64.00^{h} |
| Pool VIII₁₀₀ | | | | | | | | | | | |
| R α | 5.26 | 4.04 | 4.05 | 4.24 | 3.77 | 3.86 | 104 | 84.2 | 78.60 | 84.2 | 69.8 |
| R β | 5.31 | 4.09 | 4.1 | 3.96 | 3.77 | 3.86 | 98.2 | 78.9 | 77.20 | 82.1 | 71.2 |
| A | 5.10* | 4.14 | 4.04 | 4.22 | 3.9 | 3.807 | 110.34 | 84.08ⁱ | 79.40 | 85.0* | 69.21 |
| B | 5.13 | 4.29 | 4.12 | 4.32 | 3.86 | 3.96 | 110.24 | 82.05 | 85.12 | 84.44 | 68.86 |
| T3 | 5.162 | 4.15 | 3.96 | 4.05 | 3.73 | 3.84 | 110.02 | 84.08ⁱ | 79.40 | 86.75 | 63.95 |
| C | 5.273 | 4.33 | 4.261 | 4.344 | 3.86 | 3.96 | 109.11 | 87.63 | 83.07 | 84.19 | 68.86 |
| T2 | 5.191 | 4.14 | 3.97 | 4.08 | 3.73 | 3.85 | 109.68 | 84.21 | 79.40 | 86.92 | 63.95 |
| T3 | 5.162 | 4.15 | 3.96 | 4.05 | 3.73 | 3.84 | 109.78 | 84.11ⁱ | 79.40 | 86.87 | 63.95 |
| T5 | 5.092 | 4.14 | 3.96 | 4.12 | 3.73 | 3.84 | 110.26 | 83.96ⁱ | 79.47 | 86.68 | 63.95 |

*signal broadening or splitting due to anomerization effect;
^{a,b,c,d,e,f,g,h,i}values may have to be interchanged

Purity and Structure of Dimers

Since HPAEC confirmed that pools I₃₀ and I₁₀₀ only consisted of arabinose monomers, the first pools to be investigated in more detail were pools II₃₀ and II₁₀₀. NMR analysis of the pools II₃₀ and II₁₀₀ resulted in identical NMR data (Table 3). The component present was identified as an α-(1,5)-arabinobiose, which confirms the HPAEC results (data not shown). The NMR data are in agreement with data for α-(1,5)-arabinobiose (Cros, S.; Imberty, A.; Bouchemal, N.; Dupenhoat, C. H.; Perez, S. Biopolymers, 1994, 34, 1433-1447).

Purity and Structure of Trimers

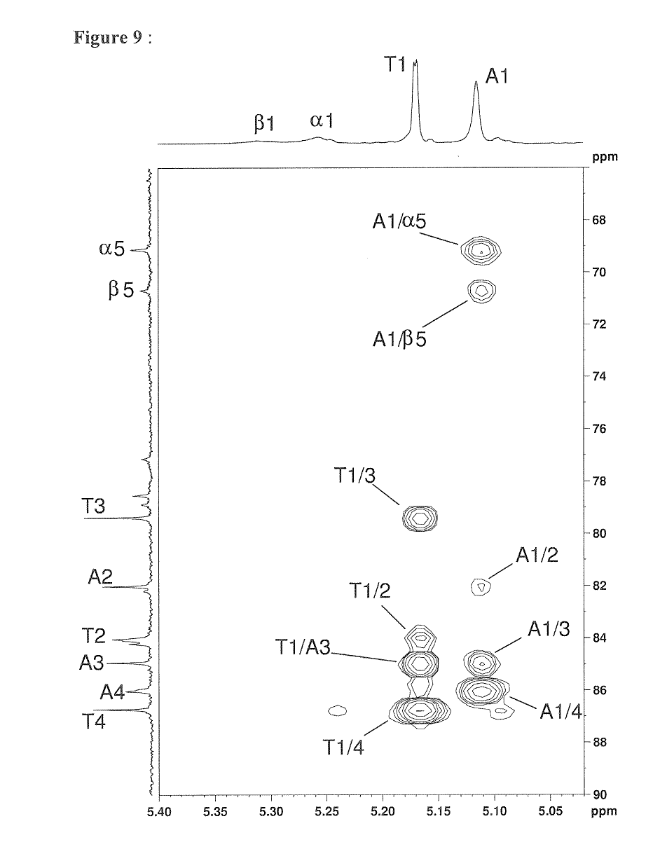

HPAEC analysis of the pools III₃₀ and III₁₀₀ reveals a major peak in the HPAEC chromatogram at 17.3 min for both samples, not co-eluting with any linear α-(1,5)-linked AOS-standard (FIG. 7B and FIG. 8B). MALDI-TOF MS indicates the presence of a pentose-oligomer with a degree of polymerization (DP) of 3 for both pools. Apparently, both pools contain the same AOS (3.1) with a purity of >90%. NMR analysis was carried out with pool III₃₀. The major component (3.1) could be assigned as a dimeric α-(1,5)-linked arabinan backbone with an α-(1,3)-linked arabinose residue at the non-reducing end (Table 4; structure 3.1) due to the following NMR characteristics: compared to the data for arabinobiose, the α-(1,3)-linkage of a third arabinose residue (Table 4, T-residue) is indicated by a cross peak in the HMBC between H-1 of this T-residue and the C-3 of the A-residue (FIG. 9, T1/A3). The downfield shift of 5.6 ppm for C-3 and the smaller upfield shifts for C-2 and C-4 of 1.4 ppm and 0.8 ppm, respectively, in the arabinose A-residue confirm the α-(1,3) linkage of the arabinose T-residue (Table 3; Capek, P.; Toman, R.; Kardosova, A.; Rosik, J. Carbohydr. Res., 1983, 117, 133-140; Dourado, F.; Cardoso, S. M.; Silva, A. M. S.; Gama, F. M.; Coimbra, M. A. Carbohydr. Polym., 2006, 66, 27-33; Cardoso, S. M.; Silva, A. M. S.; Coimbra, M. A. Carbohydr. Res., 2002, 337, 917-924). To enable the distinction between the linear α-(1,5)-linked arabino-triose (3.0) and the novel branched arabino-triose, the peak at 17.3 min received the number 3.1.

TABLE 4
Structures of arabino-oligosaccharides identified from sugar beet arabinan
(series 1 and 2), as obtained after degradation of sugar beet arabinan with the arabino-
hydrolases Abn1, Abn2 and Abn4 followed by Biogel P2 fractionation (D-30 and D-100,
respectively).

| | Structures of Identified Arabino-oligosaccharides from Sugar Beet Arabinan | |
|---|---|---|
| | Series 1 | Series 2 |
| DP3 | | 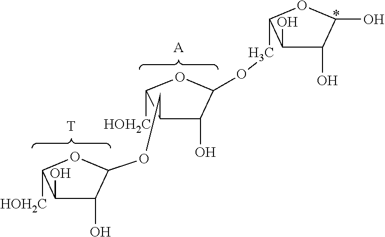 3.1 |
| DP4 | 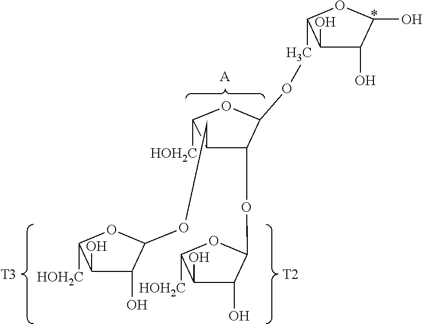 4.1 | 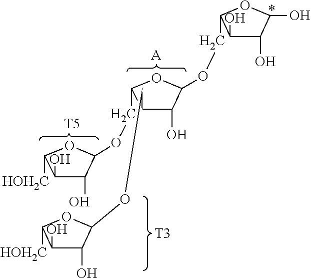 4.2 |
| DP5 | 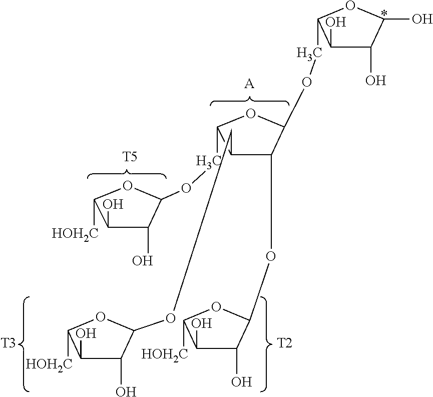 5.1 | 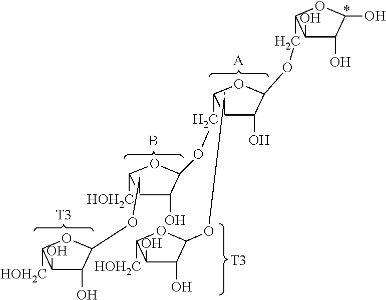 5.2 |
| DP6 | | 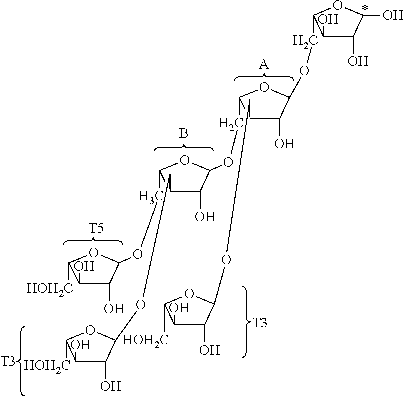 6.2 |
| DP7 | | 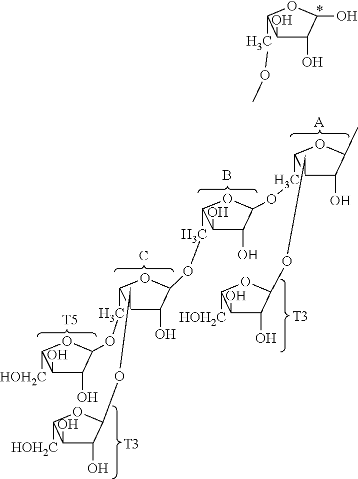 7.1 |
| DP8 | 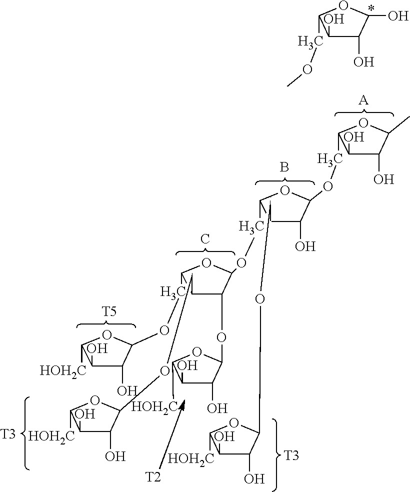 8.1 | |

Purity and Structure of Tetramers

The pools IV₃₀ and IV₁₀₀ contain pentose-oligomers of DP4 as analyzed with MALDI-TOF MS. HPAEC analysis of IV₃₀ showed one major peak, which elutes at the retention time of the linear α-(1,5)-linked arabino-tetraose (FIG. 7B; 20.1 min). To investigate if a co-eluting branched AOS is present, pool IV₃₀ has been analyzed by NMR. In the ¹³C-spectra of pool IV₃₀ the downfield shift of 5.4 ppm of the C-5 of the A-residue (Table 3 and Table 4; structure 4.2) compared to the A-residue of the component 3.1 in pool III₃₀ indicates the presence of an additional α-(1,5) linked residue. An upfield shift of 1.5 ppm for the C-4 of the A-residue (Table 3) and a HMBC cross peak between H-1 of the T5-residue and C-5 of the A-residue confirms the presence of an α-(1,5)-linked T5 residue (Table 3). Following these NMR data, the component present in pool IV₃₀ (4.2) could be assigned as a trimeric α-(1,5)-linked arabinan backbone with an α-(1,3)-linked arabinose residue at the middle arabinose unit (Table 4; structure 4.2). Thus, NMR data reveals that the main tetrameric component in the D-30 digest is a branched AOS (4.2) instead of the linear α-(1,5)-linked AOS (4.0). These two structures are apparently co-eluting in HPAEC with the separation conditions used.

According to HPAEC analysis, the pool IV₁₀₀ contains two major peaks (FIG. 8B; 16.4 min (4.1) and 20.1 min (4.0 or 4.2)) next to a number of minor peaks. Also this pool was analyzed by NMR to investigate the precise structures of the two major components. NMR analysis confirms the presence of two major components. The first component is identical to the one assigned in pool IV₃₀ (4.2). A second compound could be identified having an H-1 signal shifted downfield to 5.253 ppm of the A-residue (Table 3 and Table 4; structure 4.1). The HMBC shows a cross peak with the C-2 of this residue (arabinose-A), and from this C-2 a cross peak with another H-1 can be found in the HMBC, indicating an α-(1,2) linkage. Signals for an α-(1,3)-linked T3 residue can also be found. Compared to pool III₃₀ (3.1) the C-2 of the A-residue is shifted downfield with 6.0 ppm and the C-3 and C-1 are shifted upfield with 2.0 ppm and 1.1 ppm, respectively (Table 3), confirming the α-(1,2) linkage of T2 in pool IV₀₀ (4.1). Conclusively, the NMR data reveal the second component (4.1) as a dimeric α-(1,5)-linked arabinan backbone with an α-(1,2)-linked and an α-(1,3)-linked arabinose residue (Table 3; structure 4.1).

Purity and Structure of Pentamers

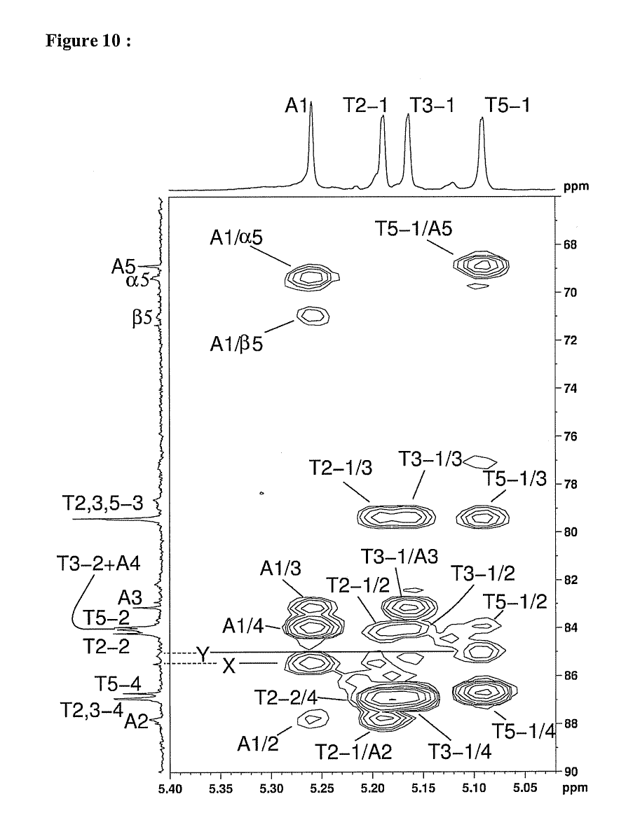

MALDI-TOF MS revealed that only pentose-oligomer(s) with DP5 are present in both pools (V₃₀ and V₁₀₀, inserted table in FIG. 7B and FIG. 8B). According to HPAEC, pool V₃₀ consists of two major oligosaccharides present in about equal amounts (FIG. 7B; 5.1 and 5.2; 20.4 min and 23.7 min, respectively), whereas pool V₁₀₀ showed the presence of only one major peak at 20.4 min (FIG. 83B). Apparently, the peak at 20.4 min represents the same component in both pools (V₃₀ and V₁₀₀; 5.1; FIG. 7B and FIG. 8B). The branched AOS 5.1 (20.4 min) elutes close to the retention time of the linear α-(1,5)-linked arabino-tetraose (FIG. 8B; 4.0; 20.1 min). The second component, present in V₃₀, represents another DP5 AOS (5.2; FIG. 8B) with substantially different retention behavior compared to 5.1, but with a similar retention behavior compared to the linear α-(1,5)-linked arabino-pentaose (5.0; 23.5 min). For further characterization of the branched AOS 5.1, the pool V₁₀₀ was analyzed by NMR as this pool contains the unknown AOS in high purity. In pool V₁₀₀ all signals for the A-residue typical for (1,2), (1,3), and (1,5)-linkages as identified in IV₃₀ and IV₁₀₀are present. Firstly, the H-1 at 5.26 ppm and the C-2 at 87.8 ppm indicates a (1,2) linkage, secondly, the chemical shift of C-3 at 83.12 ppm, which results from the combination of a downfield shift due to a (1,3) linkage and a small upheld shift due to a (1,2) linkage indicates a (1,3) linkage in combination with a (1,2) linkage, and thirdly, the C-5 at 68.88 ppm indicates a (1,5) linkage. These data are in good agreement with Capek et al¹¹. In the HMBC cross peaks between all three terminal residues (T2, T3 and T5) and the A-residue could be assigned (FIG. 10), resulting in a structure as shown in Table 4 for structure 5.1. The cross peaks denoted X is not belonging to the main component as is clear from the ¹³C-spectrum, where the signal, probably a C-4, belonging to this cross peak too low. The signal is visible in the HMBC due to the higher sensitivity of this proton detected 2D experiment and due to the high intensity of cross peaks between H-1 and C-4 in arabinoses. The value for this C-4 is indicative for a (1,2)-substituted arabinose with no (1,3)-substitution. The signal denoted with Y could indicate the presence of an arabinose with only (1,5)-substitution (compare with the B-residue in pool VII₃₀). Therefore, the structure of a minor compound in pool V₁₀₀ could be a (2,5)-substituted arabinose with an additional (1-5)-arabinose between the (2,5)-substituted arabinose and the reducing end arabinose.

Pool V₃₀ was as well subjected to NMR analysis in order to reveal the identity of the second AOS with DP5, eluting at 23.7 min (FIG. 7B, 5.2). Although a mixture of two major compounds was present in pool V₃₀, with component 5.1 (pool V₁₀₀) as one of them, it was possible to determine the structure of the second compound, because of the presence of three characteristic signals: a signal at 86.08 ppm, assigned as C-4 of the B-residue (Table 3 and Table 4) with only an α-(1,3)-linked arabinose attached to it (compare with pool III₃₀ (3.1)), and two signals at 85.18 ppm and 84.95 ppm for the A-residue and B-residue, respectively, which are typical for C-3 signals in arabinoses with only α-(1,3)-linked arabinose attached to it (Table 3 and Table 4). Due to close proximity of these two C-3 signals, the almost identical assignments for the T3 and T3-2 residues and the lower resolution of the 2D HMBC experiment, only a single combined cross peak in the HMBC confirms the two α-(1,3)-linkages. A cross peak between H-1 of the B-residue and C-5 of the A-residue connects the two α-(1,3) substituted arabinoses (data not shown). Cross peaks between the A-residue and the reducing end arabinose complete the assignment of this structure, resulting in the structure as shown in Table 4 for component 5.2.

Purity and Structure of Hexamers

HPAEC analysis of the pools VI₃₀ and VI₁₀₀ reveals the presence of each one major peak at 25.4 min and 22.6 min, respectively (FIG. 7 and FIG. 8B). As MALDI-TOF MS shows the presence of only pentose-oligomers of DP6, these oligosaccharides are assigned as 6.2 and 6.1, respectively. For further characterization, the pools were subjected to NMR analysis. The pool VI₃₀ is similar to pool V₃₀ with respect to the two (1,3)-linked residues as indicated by two signals for C-3 at 85.13 and 85.21 ppm (Table 3) together with a combined cross peak with H-1 of the T3 residue and the T3-2 residue, and a cross peak between H-1 of the B-residue and C-5 of the A-residue. In pool VI₃₀ the C-4 signal of the B-residue indicates the presence of an additional (1,5)-linked T5-residue, confirmed by a cross peak between H-1 of the T5-residue and C-5 of the B-residue in the HMBC (data not shown). Following the NMR data, component 6.2 could be assigned as tetrameric α-(1,5)-linked arabinan backbone with α-(1,3)-substitution of single arabinose residues at the two middle arabinose units as depicted in Table 4 (structure 6.2).

In pool VI₁₀₀ signals similar to those in pool IV₁₀₀ could be assigned (Table 3), indicating that the same structural element, an arabinose with (1,2) and (1,3)-linked arabinose residues attached to it, must be present. The two residues between this element and the reducing end, needed to complete the structure to 6 residues could not be assigned due to a large heterogeneity in the spectra. Thus, even though HPAEC showed only one major peak at 22.6 min, NMR analysis revealed that more than one component must be present, indicating insufficient separation of HPAEC for these compounds.

Purity and Structure of Heptamers

The pool VII₃₀ shows one major peak during HPAEC analysis (data not shown), MALDI-TOF MS analysis shows the presence of a pentose-oligomer of DP7 (7.1). For further characterization of the component 7.1, the pool VII₃₀ was analyzed by NMR. The pool VII₃₀ has all the features of pool VI₃₀, two (1,3) linked arabinose residues (T3 and T3-2, respectively) and one (1,5) linked T5 residue (Table 3). An extra signal at 85.08 ppm, assigned as C-4, indicates the presence of an additional (1,5) linked arabinose in the backbone. Two positions for this additional residue are possible: between the two (1,3)-substituted arabinoses (T3 and T3-2) or between the first (1,3)-substituted arabinose (T3) and the reducing end. The latter possibility would result in slightly different signals for the C-5 of the reducing end and is found with another non-reducing end in VIII₁₀₀ (see discussion there). The first possibility with (1,3)-linked residues on the A-residue and the C-residue, respectively, represents the main component in this pool (7.1). HMBC cross peaks between the H-1 of the C-residue and the C-5 of the B-residue confirm this assignment, resulting in the structure as depicted in Table 4 (structure 7.1).

According to HPAEC and MALDI-TOF MS analysis the pool VII₁₀₀ contains a mixture of components with DP6 and DP7, thus, no further NMR analysis was done for pool VII₁₀₀.

Purity and Structure of the Octamer

HPAEC analysis of pool VIII₁₀₀ reveals the presence of one major peak at 27.8 min, nearly at the same retention time as the linear α-(1,5)-linked arabino-heptaose (7.0). MALDI-TOF MS results show the presence of mainly DP8, thus, the unknown component represents an arabino-octaose (8.1) with a substantially different retention behavior compared to linear α-(1,5)-linked arabino-octaose (8.0; FIG. 8B). For further characterization pool VIII₁₀₀ was subjected to NMR analysis. In pool VIII₁₀₀ all signals of a triple substituted arabinose are present as was assigned for V₁₀₀ (compare pool V₁₀₀ with VIII₁₀₀ in Table 3). In the HMBC at the position of H-1 of the T3 residues two cross peaks are found with two different C-3 signals: at 83.07 ppm (C-residue, Table 3 and Table 4), characteristic for a (1,3) linkage in combination with a (1,2) linkage as mentioned in pool V₁₀₀, and at 85.12 ppm (B-residue, Table 3 and Table 4), indicating a (1,3) linkage without (1,2) substitution at the same residue (similar to IV₃₀ (4.2), V₃₀ (5.2), VI₃₀ (6.2) and VII₃₀ (7.1)). As in VII₃₀ a C-4 signal at 85.0 ppm indicates the presence of an additional (1,5)-linked arabinose residue, which is located next to the reducing end due to a clear anomerization effect of this signal (A-residue, Table 3 and Table 4). This is furthermore substantiated by cross peaks in the HMBC between the H-1 of this A-residue and the C-5 signals of the reducing end (R α/β). The chemical shifts of these C-5 carbons are slightly different for those of all structures with a (1,3) substituted A-residue, but resembles the chemical shifts found for α-(1,5)-arabinobiose, confirming the presence of an arabinose residue attached to the reducing end with no (1,3) substitution. Following all the NMR data, component 8.1, which is present in VII₁₀₀, has a structure as depicted in Table 4.

Overview of AOS Identified from Sugar Beet Arabinan

In Table 4 an overview of the structures of all identified branched AOS is given as based on extensive NMR analysis. All of them consist of an α-(1,5)-linked backbone of L-arabinosyl residues. Two main structural features could be identified among all identified AOS, varying in their type of linkages and the degree of substitution. AOS of the first series contain a structure with double substituted α-(1,2)- and α-(1,3)-linked L-arabinosyl residues (4.1, 5.1, 8.1; Table 4, series 1). An additional single substituted α-(1,3)-linked. L-arabinosyl residue may be present within the same molecule as identified in component 8.1 (Table 4). AOS of the second series carry single substituted α-(1,3)-linked arabinose(s) (Table 4; series 2). Components with either one or two α-(1,3,5)-linkages were identified (3.1, 4.2 and 5.2, 6.2, 7.2, respectively). None of the identified structures was substituted at the arabinose at the reducing end, which is contrast to the synthesized methyl 2-O, methyl 3-O— and methyl 5-O-α-L-arabinofuranosyl-α-L-arabinofuranosides as described by Kaneko et al.¹⁴. The isolated component 3.1 is similar to an earlier described feruloylated arabinose-oligosaccharide with a α-L-arabinosyl residue linked at O-3 and a ferulic acid attached at O-2 of the non-reducing end of an α-(1,5)-linked dimeric backbone of L-arabinosyl residues, which has been isolated from spinach leaves¹⁵ and sugar beet pulp¹⁶.

Almost all the AOS of the second series (Table 4; 3.1., 4.2, 5.2, 6.2 and 7.1) were only present in the D-30 digest, while the three isolated AOS belonging to the first series were only present in the D-100 digest, indicating a different degradability of the structures by the arabino-furanosidase Abn4. Further investigation concerning the mode of action and specificity of the arabino-hydrolases is currently under investigation.

### CONCLUSIONS

At least seven novel neutral branched AOS have been isolated from sugar beet arabinan after enzyme digestion with two different mixtures of the Chryosporium lucknowense arabino-hydrolases Abn1, Abn2 and Abn4. NMR analysis revealed basically two series of branched AOS varying in the type of linkage. To the best of our knowledge, this is the first time describing the isolation and characterization of these branched AOS, which may now be used for (further) characterization of arabinan-specific enzymes as well as for possible exploration of their prebiotic potential.

1. A method for hydrolyzing arabinans present in a plant biomass, comprising contacting the plant biomass with a multi-enzyme composition, wherein the multi-enzyme composition is selected from the group consisting of:
a. Abn1 (SEQ ID NO:2), Abn2 (SEQ ID NO:4), Abn4 (SEQ ID NO:6) and Abf3 (SEQ ID NO:8);
b. Abn1 (SEQ ID NO:2), Abn2 (SEQ ID NO:4), Abn4 (SEQ ID NO:6); and
c. Abn1 (SEQ ID NO:2), Abn4 (SEQ ID NO:6), and Abf3 (SEQ ID NO:8);

2. The method of claim 1, wherein the multi-enzyme composition is able to degrade at least about 70% of the arabinan present in the plant biomass to arabinose.

3. The method of claim 1, wherein the multi-enzyme composition is able to degrade at least about 80% of the arabinan present in the plant biomass to arabinose

4. The method of claim 1, wherein the multi-enzyme composition is able to degrade at least about 90% of the arabinan present in the plant biomass to arabinose.

5. The method of claim 1, wherein the enzymes are isolated from a filamentous fungus.

6. The method of claim 2, wherein the specific activity of Abn1 towards linear arabinan is from about 20 U/mg to about 30 U/mg, the specific activity of Abn2 towards linear arabinan is from about 6 U/mg to about 8 U/mg, the specific activity of Abn4 towards branched arabinan is from about 8 U/mg to about 11 U/mg, and the specific activity of Abf3 towards p-Nitrophenyl-α-arabinofuranose is from about 20 U/mg to about 30 U/mg.

7. A multi-enzyme composition comprising the enzymes Abn1 (SEQ ID NO:2), Abn2 (SEQ ID NO:4), Abn4 (SEQ ID NO:6), and Abf3 (SEQ ID NO:8); the enzymes Abn1 (SEQ ID NO:2), Abn2 (SEQ ID NO:4), and Abn4 (SEQ ID NO:6); or the enzymes Abn1 (SEQ ID NO:2), Abn4 (SEQ ID NO:6), and Abf3 (SEQ ID NO:8), wherein the multi-enzyme composition is able to degrade at least about 70%, at least about 80%, or at least about 90% of the arabinan present in sugar beet to arabinose.

8. The method of claim 1, Abn1 (SEQ ID NO:2), Abn2 (SEQ ID NO:4), and Abn4 (SEQ ID NO:6), wherein the multi-enzyme composition is used to prepare a prebiotic and is capable of degrading the arabinans in the plant biomass into linear and branched arabinanose oligomers.

9. The multi-enzyme composition of claim 7 comprising the enzymes Abn1 (SEQ ID NO:2), Abn2 (SEQ ID NO:4), and Abn4 (SEQ ID NO:6), wherein the multi-enzyme composition is able to hydrolyze arabinan present in the plant biomass into branched arabinan oligomers, and wherein the multi-enzyme composition is used to prepare a prebiotic.

10. The multi-enzyme composition of claim 9, where in the prebiotic comprises branched arabinan oligomers, wherein the branched arabinan oligomers comprise α-(1,5)-linked arabinan backbone, and a) single substituted α-(1,3)-linked arabinose monomers attached to the backbone, or b) double substituted α-(1,2,3,5)-linked arabinose monomers attached to the backbone, c) or both.

11. The method of claim 8, wherein the branched arabinan oligomers comprise α-(1,5)-linked arabinan backbone, and a) single substituted α-(1,3)-linked arabinose monomers attached to the backbone, or b) double substituted α-(1,2,3,5)-linked arabinose monomers attached to the backbone, c) or both.

12. The method of claim 1, comprising the multi-enzyme composition Abn1 (SEQ ID NO:2), Abn2 (SEQ ID NO:4), Abn4 (SEQ ID NO:6) and Abf3 (SEQ ID NO:8), wherein the multi-enzyme composition is capable of degrading the arabinans in the plant biomass into linear and branched arabinanose oligomers, and wherein the multi-enzyme composition is used to prepare a fruit juice or wine.

13. The method of claim 1, comprising the multi-enzyme composition Abn1 (SEQ ID NO:2), Abn2 (SEQ ID NO:4), Abn4 (SEQ ID NO:6) and Abf3 (SEQ ID NO:8), wherein the multi-enzyme composition is capable of degrading the arabinans in the plant biomass into linear and branched arabinanose oligomers and wherein the multi-enzume composition is used for the saccharification of a plant biomass.

14. The method of claim 12, wherein the multi-enzyme composition further comprises one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase and wherein the plant biomass comprises pectins, hemi-celluloses and/or celluloses.

15. The method of claim 13, wherein the multi-enzyme composition further comprises one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase and wherein the plant biomass comprises pectins, hemi-celluloses and/or celluloses.

16. A recombinant micro-organism, wherein the microorganism is genetically modified to express Abn1 (SEQ ID NO:2), Abn2 (SEQ ID NO:4), Abn4 (SEQ ID NO:6), Abf3 (SEQ ID NO:8), or a combination thereof.

17. The recombinant micro-organism of claim 16, wherein the micro-organism expresses one or more of the following enzymes: endo-polygalacturonase, pectin/pectate lyase, pectin methyl esterase, endo-glucanase, cellobiohydrolase, β-glucosidase, xylanase, β-xylosidase and ferulic acid esterase.

18. The recombinant micro-organism of claim 16, wherein the micro-organism is a filamentous fungus.

19. The recombinant micro-organism of claim 17, wherein the micro-organism is a filamentous fungus.


SEQUENCE LISTING (12 sequences)

SEQ ID NO: 1 - DNA, 1504 bp, Chrysosporium lucknowense
  CDS 296..565
  CDS 650..1240
  CDS 1351..1452
acaccctcacgacaagtcgcaacctcgatttcccgtgtgcccggagcttggcaacgcgtcacgacccatagttagtagtatcgtcaagggccgtctcgctaaaagtagcgtggtttcagaaaccgtttatttagcgaccttaaataattattattgattatctcccgacaacctccgggcctcgtcggcgggtttctgtctaactgctgctgcaaacttcaggcggctgtccgtcgtgttcggtcattctcaccttgccagctttggctaattaccttaccttgctctaccaacgatgaagagatccggccttctggggacggtggctcttctgttcccggccctcgtttctgcctatgcgctcccgggcgaatgtactggagtctgcaacaactcccacgacccgtcaatcatccgccgcgcagatgggacgtacttccgcttctcgactggtggcaagatcgccattcacacggcgccgagcctgaccggcccgtggacctataggggcgccgccttgccggccgggtcctccatcaacttggacgggaaccaggacctctgggtaagaagaacaaggcgttcacggcccgcagcctggatgtaatttatttatgggtgatgatgatgttgacgacgagctcgccaggcccccgatgttgcacaggtcggagaccagtactacctgtactactccgtcagcaccttcggcgtccagaactcggccatcggcatagcccgctcatcgagcctggacgccggcacctggaccgacctcgggtctaccggtgtcacctccagcacgggctcggcgtacaacgccatcgacggcaacctgatcaacgagccggggaccagcaactacttcctcacgtttggcagcttctggaacggcatccatcgcgtccgcatgacgcccaccaagacgaacggcaacgtgtaccaggtcgcctttgatcccaacgacccggccatggaaggcccgaccgtcttcaagtacggcgactactactaccttttcttctccaagggcacgtgctgcggctacgaccagaacaggccgcccgccggacaggagtacaggatcatggtgtgcaggtcgacctcgcccaccggccccttcgaggacagggacggcaagtcgtgccgctcgggcggtggaactctggtgctcccgtctcacgactgggtgtacggccccggcggccaaggtgtctaccaggaccccgaacacggaccggttagttccccgtcctttctcccgtacgggcctagcattcagcatcggggatcgagcatcaagcatcaccctgcttgaacccatctcttgctaacgctcttccttcccaggtcttgtactaccattatgttgatactcgcattggctatgcggacggccagaagaagtttggttggaatcgcatcgacttttcaagcggttggccggttgtctgagaaacccgagacgcttgacttgtttatttacaggtattaccggtattaa

SEQ ID NO: 2 - protein, 321 aa, Chrysosporium lucknowense
MKRSGLLGTVALLFPALVSAYALPGECTGVCNNSHDPSIIRRADGTYFRFSTGGKIAIHTAPSLTGPWTYRGAALPAGSSINLDGNQDLWAPDVAQVGDQYYLYYSVSTFGVQNSAIGIARSSSLDAGTWTDLGSTGVTSSTGSAYNAIDGNLINEPGTSNYFLTFGSFWNGIHRVRMTPTKTNGNVYQVAFDPNDPAMEGPTVFKYGDYYYLFFSKGTCCGYDQNRPPAGQEYRIMVCRSTSPTGPFEDRDGKSCRSGGGTLVLPSHDWVYGPGGQGVYQDPEHGPVLYYHYVDTRIGYADGQKKFGWNRIDFSSGWPVV

SEQ ID NO: 3 - DNA, 1716 bp, Chrysosporium lucknowense
  CDS 113..413
  CDS 502..1334
ctttataacaaggcccgacccgagctcacgacagcgacgccgtgactctttctctgttggttaggccaagtcattattacccgaaccagagagagacgtctagacggtcaatatgaagtggtccacgctcatcccctctgtccttgttccccttgtctcggctgctcccaaggagcgggaggagaagcccgagaccttctcgcagcgcgttatcttcacgccgcccgacaactacaccgacccgcgcgtgctgtacgcccgcaccgcccagttccgggacggcaccctgctcgcgacctgggagaattacagcccggagccccctccggtctacttccccatctaccgctccactgacggcggctacaactggaaggaaatctcgcgtgtcgaggacacggccaacggattgggtaggggtttttttttttttaacgtttcgtggattcggcggtccgagggcgattactaacaacaccgcctctccccccccccctccaggcctccgataccagccgttcctctacatcctggatgagtcctttgccggcttccccaaggacacggtcctgctggccgggagctcgatccccacggacctctcaagcacccagatcgacctgtatgcctcgacggaccgcggcgtgacgtgggagtttgtctcgcacatcgcggccggcggcgtggccatccccaacaacggcgagacccctgtctgggagccgttcctgatgcgcaacggagacacgctcatctgctactactcggaccagcgcgagaacacgacgtacggccagaagatggtgcaccagaccacctcggacctgcgcacctggggccccgtcgtcaacgacgtcacgtcgcccgtctacgaggagcgcccgggcatgccgatcgtcgccaagctgcccaacggcaagtacatcatgacgtacgagcacggcggcagccccgtgctgagcggctaccagttccccgtccactacaaggtcgtgtcggatcccgagcggttcggccccgccaccggcgtgccgctccggaccaccgacggctacatcccgaccggctcgccctacgtcgtctggagccccgtcggcggggagaacggcacggtgatcgccagcgcccacagctcggccgacatctacatcaacacgggcctcgccgagcccgggagcacctggaggcgcgtcgccacgcccgagaagaacgcctacacgcgccatctccgcgtcctcaacgaccctaccaagctgctcatcatgggcgccggccagttgccgccaagcaccaccaaccaggtggagctgagcgtgattgacatcagcaacctgtaaaggggggtggggggggggggggaccggcaaagttctagagaacagtgggtgggtgctggatagggtagagcgactccgttttgagattttgtcaaaaacaacactcgtaagagcaatttccctcgcggtgttgagaaatcaggtattgacacagcgaagcatctcttcgtgatggggccatgacatgcggtggaaccttctcgtgcggtgattgacggatctctatgcattatgaaggagttcgccgcgtgctaggtagcatacaagcggcttagctcagagccacgctccagatcccccaagcgtagcggcgcatatcgtgttgttatatatagtgcccttgacccctctccatatattatacatacatatctataaa

SEQ ID NO: 4 - protein, 378 aa, Chrysosporium lucknowense
MKWSTLIPSVLVPLVSAAPKEREEKPETFSQRVIFTPPDNYTDPRVLYARTAQFRDGTLLATWENYSPEPPPVYFPIYRSTDGGYNWKEISRVEDTANGLGLRYQPFLYILDESFAGFPKDTVLLAGSSIPTDLSSTQIDLYASTDRGVTWEFVSHIAAGGVAIPNNGETPVWEPFLMRNGDTLICYYSDQRENTTYGQKMVHQTTSDLRTWGPVVNDVTSPVYEERPGMPIVAKLPNGKYIMTYEHGGSPVLSGYQFPVHYKVVSDPERFGPATGVPLRTTDGYIPTGSPYVVWSPVGGENGTVIASAHSSADIYINTGLAEPGSTWRRVATPEKNAYTRHLRVLNDPTKLLIMGAGQLPPSTTNQVELSVIDISNL

SEQ ID NO: 5 - DNA, 2033 bp, Chrysosporium lucknowense
  CDS 494..777
  CDS 870..937
  CDS 1023..1446
  CDS 1576..1759
atattaatagctgctggaatccttcgctttaccaactcccgcagactgttgcatcgttccctcgtggccagttcgcgcaacgggaatcaacggagaacccggggaataataccccatgcgcctagccagagccgcgatgcgcgccaatagacatgcacgtttcagcgtgtcagaacgggagttcggcggttttgtttccgcaagtgacactgcccccgatcggaatccacttcgatcaacgagagagtttttccgacgtgcatacatgctactacatggtacgagcacgaggaaagctatgttacaagttgtagtctgtaagccgaggtcttatagccatgtagctcacaagatgtgtcaagggagcgcctaatagttctgtaattaataaaccctcacccgggtgctccagcttgaatgaaatactactgcgttcctactctatccttggtaaccctcatctatgccaaattctgtctttaactagcacaagatgaagggtgtttccatcgcaggggccttctcgggcctccttagcttggcctcagctgcctctttctcgaatcctctcaaacccttcaacgggtcagatcctcatatgacctactatgatgggtactattatctgatgacgacaacatggaccgacctgcagataacccgagccaagaccttagagggcctcaaaaacggagagaccagggtggtttggactgactcggatccctcccgctgctgcaatgtctgggcacccgagcttcacaagattgacaacaagtaagtggtcaccatcagccccagcatggttattaattaaactctctttatcaatgataggaggaggttgggctaaaagaaaacatcaataggtggtacatctactacacagctggaaacaaccagaacctagatggccagcggtctcatgtcctagaaggttagtgaaagagcagctgcctgctattttgagcaattaggtctggtaggttctcgaggagactgacttatatacacaaatctaggtggttctgacccgtggggccagtactcctatgccggacagctcacccaagactggggcatcgacggtacgattctccgcgtccagaaccagaactactttgtttggtcctgcttctcggacggtatccagtccctctgcattgcgcctttgcagtcaccgacttcgattggctctccccgagtgctctcgcaaccgctcctggactgggaacgggtcgacactccggttaatgaggggccggcaccgctctaccatgatggaaaaatcttcctcgcctatagtgccagtttctgctggacacctagctaccaactcggcctccttacttacaacgggggggatccgactgcagccagcagctggacaaagacggggcccttcttcagctcggcaaatggaaactacggtccgggacacaattcgtgagtacctccaatagagctcgagcatttccccatcattaccgccgcccctgatcgtgagtcacggggtgtgtgctgcacgctgcctggcagtaggcgctgtacacatactgacctgatcggacccaggtttgtcaatagcccggacggcaccgagatctggaacatctaccatgcgacctccatctcaactggtaattgtgatggcaacaggtacaccgcggccaagaaggtgacctggaattcggatggctctcccaactttggcagccccgatcccctaggcgttgtcactcctgggccctcgggggagtaacggtctccggtggtcctagttataataataacttccgcagtttttagcacggacactgtaacttcattcgccttgttggtaattatgtgtcaccgtgtggaaggcagatcatgaaggtacccagcctatgaaccgtcgacgatgcgtgcatttccatcaagaagaggaaggacctgggaaagacacaatgtacgctatagctgatgatgtccattatggaccaccaacttcgcagctgtttctgccatgatatggtgaaccattgcgggaa

SEQ ID NO: 6 - protein, 320 aa, Chrysosporium lucknowense
MKGVSIAGAFSGLLSLASAASFSNPLKPFNGSDPHMTYYDGYYYLMTTTWTDLQITRAKTLEGLKNGETRVVWTDSDPSRCCNVWAPELHKIDNKWYIYYTAGNNQNLDGQRSHVLEGGSDPWGQYSYAGQLTQDWGIDGTILRVQNQNYFVWSCFSDGIQSLCIAPLQSPTSIGSPRVLSQPLLDWERVDTPVNEGPAPLYHDGKIFLAYSASFCWTPSYQLGLLTYNGGDPTAASSWTKTGPFFSSANGNYGPGHNSFVNSPDGTEIWNIYHATSISTGNCDGNRYTAAKKVTWNSDGSPNFGSPDPLGVVTPGPSGE

SEQ ID NO: 7 - DNA, 2693 bp, Chrysosporium lucknowense
  CDS 147..298
  CDS 366..836
  CDS 946..1029
  CDS 1123..1701
  CDS 1795..1987
  CDS 2125..2607
gtttataagatggctaattacctccaccataacacacctcttagactctggacacggctgcttgttgtgcttgtatgtacatacttgcgctgtgggcaagcaaaccgaagctcctccgttcagttagactccgtctgctgtccaagatggcatctctctcactccgggctctcctcgggtttgttggcgccacggcagcggtgaccgtgaccgtggccagctctggcggacatgccacttctccgtacatgtacggtatcatgtttgaggacattaaccagagtggcgatggtggcttgtaagtgctggttcgcctctgacgcaaaggggtgggacgttcccgagctgagaagataccctcccaggtacgccgagttgatctcgaaccgggcctttcaaaacgagtcgctaaagtcctggagcgccgtgggcgacagcacgctggcgctcgacaccaccacgccgctgtcgtgggccctgcctcgctcggtccgggtcaaggggggaaagggcaaggccggcctgaggaactcgggctactggggcatcgacgtcaagcacaccgacacgtacaccggcagcttctactcgtacggcgagtacaggggtagcttcaccgcctccctcgtgtccgacatcaccaacgagacgctggccatcaccaagatcaagtccaagtcggtcgccaatgcctggacgaagcacgagttcaagttgcacccgaccaagagcgcggcgaacagcaacaacagcttcgtcctcgagttccgccccagccatgagacggagctcaagttcaacctgatcagcctcttccccccaacatacaacaacaggtgagctagctccctcccttccaatccaagtctttgtctctcgtccggcagaccgttttccccaccagagcttcttctgttctgacactacatgtcccatgtctctcaggccgaacggtatgcgccgggacttgatggagaagctccgtgacctcaagccgagtttccttcggatacctggcggcaacaacctgtaagccatcctccatcttccaagtcggcacacgtgtgtggcgtagacttttgggcggccctccctgacaccaagtgatttaaaacaaaacagcgagggcaactttgccggcaaccactggaactggtccgccacgatcggcccgctgattgagcggcccggccgcatgggcgtgtgggggtacgagaacacggatgggctcggcctggtcgagtacatgcactgggcggaggacttgggcgtcgacgtcgtgctcggggtggccgcgggtctctacctgaacggcgacgtcgttcccgaggaggagctggacttcttcgtgcaggacgcgctcaacgagctcgagttcctgatgggcgacgtctcgactccgtacggcgccctgcgcgccaagctcggctaccccaggccgtggaagatcaagttcgtcgagatcggcaacgaagacaacctctggggcggcctcgactcgtacaagagctaccgactgagggccttctacgacgccatcaaggccaagtacccggacatcttcatcttctcatcgaccgacgaggtcgtctacaagaagtcgggccgcgattaccacaagtacacgcgtccggactactccgtctcccagttccacctgtttgacaactggactgcggagcaccccgtcctcatcggcgagtgagtattaattgattaattagtcgagactcacggcgctctctccttgcccacattccggctgtgagctgacgagagtgaccttttccgcaggtacgccaccatccagaacaacacgggcaagctcgaggacacggactgggacgcgcccaagaacgcgtggtccaactggatcggctccgtcgccgaggccgtcttcctgctcggcgccgagcgcaacgccgacaagatctggggcaccgccttcgcgccgctgttccagaacctcaacaactaccagtgggccgtaggtcactctcctttcccccctttccccccctttccctccccacttcccccttttcaccaacgggttggaggtgggtcgtcgaatgatacatgcggtggctgatgacacggggttcgacgacggcacgaacacagcccgacctgatctccttcaccgccgacccggccgacaccaccgtcagcgtgtcgtaccccatcatccagctcttctcggagcaccgcatcacccacaccctcccggccgcgtccccggacgggttcggcccgctgtactgggccgccggtcgcggcggcctcggcggcggcggcggcggcggcggcggcggcggcggtcacgtgtcctcctcctcctcctcctacatcctcaaggtggccgtttacaacagcaccgggtccgaggacgtgccggtgagcgtgcgcttcgagggcctcccgaagggcagccccgcaaccctgacggtgctgacggcggccggcagcgacccctgggccaagaacacgcctggccgtcccgacgccgtcaacaagaccatccagcacctgcgggctggcaagcacggcgagatcgacttcagcctgcccgacctgtcggtcgccgtcttggttgcgcgggggaagtgatgcaggtccttttttttttctctctctgtttcttccctgttagatattcaaaggagagggtagggggtggagggaaaagaaaa

SEQ ID NO: 8 - protein, 654 aa, Chrysosporium lucknowense
MASLSLRALLGFVGATAAVTVTVASSGGHATSPYMYGIMFEDINQSGDGGLYAELISNRAFQNESLKSWSAVGDSTLALDTTTPLSWALPRSVRVKGGKGKAGLRNSGYWGIDVKHTDTYTGSFYSYGEYRGSFTASLVSDITNETLAITKIKSKSVANAWTKHEFKLHPTKSAANSNNSFVLEFRPSHETELKFNLISLFPPTYNNRPNGMRRDLMEKLRDLKPSFLRIPGGNNLEGNFAGNHWNWSATIGPLIERPGRMGVWGYENTDGLGLVEYMHWAEDLGVDVVLGVAAGLYLNGDVVPEEELDFFVQDALNELEFLMGDVSTPYGALRAKLGYPRPWKIKFVEIGNEDNLWGGLDSYKSYRLRAFYDAIKAKYPDIFIFSSTDEVVYKKSGRDYHKYTRPDYSVSQFHLFDNWTAEHPVLIGEYATIQNNTGKLEDTDWDAPKNAWSNWIGSVAEAVFLLGAERNADKIWGTAFAPLFQNLNNYQWAPDLISFTADPADTTVSVSYPIIQLFSEHRITHTLPAASPDGFGPLYWAAGRGGLGGGGGGGGGGGGHVSSSSSSYILKVAVYNSTGSEDVPVSVRFEGLPKGSPATLTVLTAAGSDPWAKNTPGRPDAVNKTIQHLRAGKHGEIDFSLPDLSVAVLVARGK

SEQ ID NO: 9 - DNA, 963 bp, Chrysosporium lucknowense
atgaagagatccggccttctggggacggtggctcttctgttcccggccctcgtttctgcctatgcgctcccgggcgaatgtactggagtctgcaacaactcccacgacccgtcaatcatccgccgcgcagatgggacgtacttccgcttctcgactggtggcaagatcgccattcacacggcgccgagcctgaccggcccgtggacctataggggcgccgccttgccggccgggtcctccatcaacttggacgggaaccaggacctctgggcccccgatgttgcacaggtcggagaccagtactacctgtactactccgtcagcaccttcggcgtccagaactcggccatcggcatagcccgctcatcgagcctggacgccggcacctggaccgacctcgggtctaccggtgtcacctccagcacgggctcggcgtacaacgccatcgacggcaacctgatcaacgagccggggaccagcaactacttcctcacgtttggcagcttctggaacggcatccatcgcgtccgcatgacgcccaccaagacgaacggcaacgtgtaccaggtcgcctttgatcccaacgacccggccatggaaggcccgaccgtcttcaagtacggcgactactactaccttttcttctccaagggcacgtgctgcggctacgaccagaacaggccgcccgccggacaggagtacaggatcatggtgtgcaggtcgacctcgcccaccggccccttcgaggacagggacggcaagtcgtgccgctcgggcggtggaactctggtgctcccgtctcacgactgggtgtacggccccggcggccaaggtgtctaccaggaccccgaacacggaccggtcttgtactaccattatgttgatactcgcattggctatgcggacggccagaagaagtttggttggaatcgcatcgacttttcaagcggttggccggttgtc

SEQ ID NO: 10 - DNA, 1134 bp, Chrysosporium lucknowense
atgaagtggtccacgctcatcccctctgtccttgttccccttgtctcggctgctcccaaggagcgggaggagaagcccgagaccttctcgcagcgcgttatcttcacgccgcccgacaactacaccgacccgcgcgtgctgtacgcccgcaccgcccagttccgggacggcaccctgctcgcgacctgggagaattacagcccggagccccctccggtctacttccccatctaccgctccactgacggcggctacaactggaaggaaatctcgcgtgtcgaggacacggccaacggattgggcctccgataccagccgttcctctacatcctggatgagtcctttgccggcttccccaaggacacggtcctgctggccgggagctcgatccccacggacctctcaagcacccagatcgacctgtatgcctcgacggaccgcggcgtgacgtgggagtttgtctcgcacatcgcggccggcggcgtggccatccccaacaacggcgagacccctgtctgggagccgttcctgatgcgcaacggagacacgctcatctgctactactcggaccagcgcgagaacacgacgtacggccagaagatggtgcaccagaccacctcggacctgcgcacctggggccccgtcgtcaacgacgtcacgtcgcccgtctacgaggagcgcccgggcatgccgatcgtcgccaagctgcccaacggcaagtacatcatgacgtacgagcacggcggcagccccgtgctgagcggctaccagttccccgtccactacaaggtcgtgtcggatcccgagcggttcggccccgccaccggcgtgccgctccggaccaccgacggctacatcccgaccggctcgccctacgtcgtctggagccccgtcggcggggagaacggcacggtgatcgccagcgcccacagctcggccgacatctacatcaacacgggcctcgccgagcccgggagcacctggaggcgcgtcgccacgcccgagaagaacgcctacacgcgccatctccgcgtcctcaacgaccctaccaagctgctcatcatgggcgccggccagttgccgccaagcaccaccaaccaggtggagctgagcgtgattgacatcagcaacctg

SEQ ID NO: 11 - DNA, 960 bp, Chrysosporium lucknowense
atgaagggtgtttccatcgcaggggccttctcgggcctccttagcttggcctcagctgcctctttctcgaatcctctcaaacccttcaacgggtcagatcctcatatgacctactatgatgggtactattatctgatgacgacaacatggaccgacctgcagataacccgagccaagaccttagagggcctcaaaaacggagagaccagggtggtttggactgactcggatccctcccgctgctgcaatgtctgggcacccgagcttcacaagattgacaacaagtggtacatctactacacagctggaaacaaccagaacctagatggccagcggtctcatgtcctagaaggtggttctgacccgtggggccagtactcctatgccggacagctcacccaagactggggcatcgacggtacgattctccgcgtccagaaccagaactactttgtttggtcctgcttctcggacggtatccagtccctctgcattgcgcctttgcagtcaccgacttcgattggctctccccgagtgctctcgcaaccgctcctggactgggaacgggtcgacactccggttaatgaggggccggcaccgctctaccatgatggaaaaatcttcctcgcctatagtgccagtttctgctggacacctagctaccaactcggcctccttacttacaacgggggggatccgactgcagccagcagctggacaaagacggggcccttcttcagctcggcaaatggaaactacggtccgggacacaattcgtttgtcaatagcccggacggcaccgagatctggaacatctaccatgcgacctccatctcaactggtaattgtgatggcaacaggtacaccgcggccaagaaggtgacctggaattcggatggctctcccaactttggcagccccgatcccctaggcgttgtcactcctgggccctcgggggag

SEQ ID NO: 12 - DNA, 1962 bp, Chrysosporium lucknowense
atggcatctctctcactccgggctctcctcgggtttgttggcgccacggcagcggtgaccgtgaccgtggccagctctggcggacatgccacttctccgtacatgtacggtatcatgtttgaggacattaaccagagtggcgatggtggcttgtacgccgagttgatctcgaaccgggcctttcaaaacgagtcgctaaagtcctggagcgccgtgggcgacagcacgctggcgctcgacaccaccacgccgctgtcgtgggccctgcctcgctcggtccgggtcaaggggggaaagggcaaggccggcctgaggaactcgggctactggggcatcgacgtcaagcacaccgacacgtacaccggcagcttctactcgtacggcgagtacaggggtagcttcaccgcctccctcgtgtccgacatcaccaacgagacgctggccatcaccaagatcaagtccaagtcggtcgccaatgcctggacgaagcacgagttcaagttgcacccgaccaagagcgcggcgaacagcaacaacagcttcgtcctcgagttccgccccagccatgagacggagctcaagttcaacctgatcagcctcttccccccaacatacaacaacaggccgaacggtatgcgccgggacttgatggagaagctccgtgacctcaagccgagtttccttcggatacctggcggcaacaacctcgagggcaactttgccggcaaccactggaactggtccgccacgatcggcccgctgattgagcggcccggccgcatgggcgtgtgggggtacgagaacacggatgggctcggcctggtcgagtacatgcactgggcggaggacttgggcgtcgacgtcgtgctcggggtggccgcgggtctctacctgaacggcgacgtcgttcccgaggaggagctggacttcttcgtgcaggacgcgctcaacgagctcgagttcctgatgggcgacgtctcgactccgtacggcgccctgcgcgccaagctcggctaccccaggccgtggaagatcaagttcgtcgagatcggcaacgaagacaacctctggggcggcctcgactcgtacaagagctaccgactgagggccttctacgacgccatcaaggccaagtacccggacatcttcatcttctcatcgaccgacgaggtcgtctacaagaagtcgggccgcgattaccacaagtacacgcgtccggactactccgtctcccagttccacctgtttgacaactggactgcggagcaccccgtcctcatcggcgagtacgccaccatccagaacaacacgggcaagctcgaggacacggactgggacgcgcccaagaacgcgtggtccaactggatcggctccgtcgccgaggccgtcttcctgctcggcgccgagcgcaacgccgacaagatctggggcaccgccttcgcgccgctgttccagaacctcaacaactaccagtgggcccccgacctgatctccttcaccgccgacccggccgacaccaccgtcagcgtgtcgtaccccatcatccagctcttctcggagcaccgcatcacccacaccctcccggccgcgtccccggacgggttcggcccgctgtactgggccgccggtcgcggcggcctcggcggcggcggcggcggcggcggcggcggcggcggtcacgtgtcctcctcctcctcctcctacatcctcaaggtggccgtttacaacagcaccgggtccgaggacgtgccggtgagcgtgcgcttcgagggcctcccgaagggcagccccgcaaccctgacggtgctgacggcggccggcagcgacccctgggccaagaacacgcctggccgtcccgacgccgtcaacaagaccatccagcacctgcgggctggcaagcacggcgagatcgacttcagcctgcccgacctgtcggtcgccgtcttggttgcgcgggggaag